(19) United States
(12) Patent Application Publication
(10) Pub. No.: US 2025/0006323 A1
(43) Pub. Date: Jan. 2, 2025

(54) PHARMACEUTICAL PACKAGING AND METHOD FOR DELIVERY OF SAME

(71) Applicant: Daya Medicals, Inc., Reno, NV (US)

(72) Inventors: Kantilal Kasan DAYA, Reno, NV (US); Justin K. DAYA, Reno, NV (US)

(21) Appl. No.: 18/827,958

(22) Filed: Sep. 9, 2024

Related U.S. Application Data
(63) Continuation of application No. 18/339,455, filed on Jun. 22, 2023, now abandoned.
Application No. 18/339,455 is a continuation of application No. 17/031,013, filed on Sep. 24, 2020, now abandoned.
Application No. 17/031,013 is a continuation of application No. 15/955,084, filed on Apr. 17, 2018, now abandoned.
Application No. 15/955,084 is a continuation of application No. 14/645,034, filed on Mar. 11, 2015, now abandoned.
Application No. 14/645,034 is a continuation of application No. 12/570,427, filed on Sep. 30, 2009, now abandoned.
Application No. 12/570,427 is a continuation-in-part of application No. 11/348,786, filed on Feb. 7, 2006, now abandoned.
(60) Provisional application No. 61/271,292, filed on Jul. 20, 2009.
(60) Provisional application No. 60/742,576, filed on Dec. 6, 2005.
(60) Provisional application No. 60/736,355, filed on Nov. 14, 2005.

(51) Int. Cl. G16H 10/60; A61J 1/03; A61J 7/00; A61J 7/04; B65D 25/04; G06Q 10/06; G06Q 40/08; G06Q 50/22; G06Q 99/00; G16H 20/10; G16H 20/13; G16H 50/20; G16H 50/30; G16Z 99/00

(52) U.S. Cl. CPC G16H 10/60; A61J 1/03; B65D 25/04; G06Q 10/06; G06Q 40/08; G06Q 50/22; G06Q 99/00; G16H 20/10; G16H 20/13; G16H 50/20; G16H 50/30; G16Z 99/00; A61J 1/035; A61J 7/0084; A61J 7/049; A61J 2200/30; Y04S 10/50

(57) ABSTRACT

A disease management system including: a Diagnostic Module, which provides access to patient information and scientific guidelines for patient treatment; a Diagnostic Interpretive Module, which provides tools to evaluate risk of particular diseases or conditions based on patient information and an evaluative methodology; a Prescriptive Module, which is used to recommend, select, and/or evaluate one or more treatment regimens based on patient information and guidelines; a Dispensing Module, which evaluates a patient's compliance with a treatment regimen; and/or a Feedback and Patient Management Module, which gathers compliance information and evaluates efficacy of a treatment regimen for a patient. In embodiments of the 10 subject invention, some or all of the modules described can communicate to manage a disease, medical condition, and/or health problem in a patient.

### CROSS-REFERENCE TO RELATED APPLICATIONS

This application is a continuation of U.S. application Ser. No. 18/339,455, filed Jun. 22, 2023, which is a continuation of U.S. application Ser. No. 17/031,013, filed Sep. 24, 2020, now abandoned, which is a continuation of U.S. application Ser. No. 15/955,084, filed Apr. 17, 2018, now abandoned, which is a continuation of U.S. application Ser. No. 14/645,034, filed Mar. 11, 2015, now abandoned, which is a continuation of U.S. application Ser. No. 12/570,427, filed Sep. 30, 2009, now abandoned, which claims the benefit of U.S. Provisional Application Ser. No. 61/271,292, filed Jul. 20, 2009, and is a continuation-in-part of U.S. application Ser. No. 11/348,786, filed Feb. 7, 2006, now abandoned; which claims the benefit of U.S. Provisional Application Ser. Nos. 60/742,576, filed Dec. 6, 2005, and 60/736,355, filed Nov. 14, 2005, which are hereby incorporated by reference in their entirety, including any figures, tables, or drawings, to the extent they are not inconsistent with the teachings explicitly set forth herein.

### BACKGROUND OF INVENTION

Modifiable major health risk factors can be controlled with appropriate medicaments. It is well known that three major risk factors—serum cholesterol level, blood pressure, and smoking—increase the incidence of coronary heart disease (CHD) and related end points. Long-term studies have amassed extensive data on the relationships of major coronary-cardiovascular, cerebrovascular and diabetic sequelae risk factors—particularly serum cholesterol level, blood pressure (BP), cigarette smoking, and the consequent uncontrolled action of platelets—with incidence of coronary heart disease (CHD), stroke and cardiovascular disease (CVD) to mortality from these causes. These relationships have been characterized as strong, continuous, graded, consistent, independent, predictive, and etiologically significant for CHD, CVD, and diabetic sequelae.

Hypertension is a well documented risk factor for coronary artery, cerebrovascular, renovascular, and ocular vascular diseases. Unfortunately, hypertension remains vastly untreated. High blood pressure is also a major risk factor for stroke and heart disease.

Studies have also shown that reducing total/gross cholesterol levels, particularly Low Density Lipoproteins (LDL), Very Low Density Lipoproteins (VLDL), and an increase in High Density Lipoproteins (HDL) in patients, ranging from pediatric, teen and adult ages, inhibits the onset of heart disease in apparently healthy persons. Treatment of relatively high risk men with clearly elevated cholesterol levels significantly reduced risk of heart attack and death from heart disease. Persons with heart disease or those deemed to be at high risk for stroke or heart attack should seriously consider treatment if their LDL cholesterol level is greater than about 130 mg/dl. Reports have indicated that reducing LDL cholesterol and total blood cholesterol can reduce the incidence of coronary heart disease and heart attacks in men at high risk because of significant amounts of plasma cholesterol.

Several other studies have shown that treating abnormal lipid levels can reduce cardiovascular morbidity and mortality.

A patient can reasonably control his or her elevated blood pressure, abnormal cholesterol level, elevated triglycerides, blood glucose levels, tobacco use and the consequent aberrant adherent behavior of platelets, uncontrolled diabetes, obesity, and physical activity. Patients cannot control their age, family history of early heart disease (having a father or brother diagnosed with heart disease before age 55 or having a mother or sister diagnosed before age 65), and strongly imbedded habits, such as smoking.

In the healthcare community, there is consistent agreement that modifiable risk factors responsible for the cardiovascular and stroke epidemic in the Western World include: elevated uncontrolled blood pressure (>115/75 mm Hg), elevated cholesterol (>130 mm L), elevated triglycerides, cigarette smoking and its consequent aberrant adherent stimulation of platelets, elevated levels of C-Reactive proteins, obesity, lack of exercise, uncontrolled diet, and absence of consumption of fruits and vegetables.

In the past decade the concept has evolved that the intensity of risk factor management should be adjusted according to the severity of estimated risk. Global risk patient assessment is the estimation of absolute risk based on the summation of risks contributed by each risk factor. Several methods have been used to sum risks. The Framingham, Mass. researchers recently proposed a method in which the continuous relationship between risk-factor intensity and coronary risk is employed. Framingham scoring uses only the “standard” risk factors (smoking, blood pressure, total serum cholesterol, HDL cholesterol, blood glucose and age). But, conditional and predisposing risk factors are not used in the Framingham risk equation because of lack of evidence for a strong, independent contribution to CHD risk prediction. Several of the conditional and predisposing risk factors may contribute to the development of CHD. Thus, their detection and therapeutic modification may be appropriate in some patients.

The past decade has witnessed major strides in the prevention of CHD through modification of its causes. The most dramatic advance has been the demonstration in a “mega International” study of 17,800 men and women with normal cholesterol levels that found rosuvastatin (statin use) cuts deaths from heart attacks and strokes of healthy individuals (primary prevention). Other dramatic advances have indicated that aggressive medical therapy can substantially reduce the likelihood of recurrent major coronary syndromes in patients with established CHD (secondary prevention).

A similar potential exists for risk reduction in patients without established CHD (primary prevention). However, the risk status of persons without CHD varies greatly, and this variability mandates a range in the intensity of interventions. Effective primary prevention thus requires an assessment of risk to categorize patients for selection of appropriate interventions. Some of the major and independent risk factors for CHD are cigarette smoking of any amount, elevated blood pressure, elevated serum total cholesterol and low-density lipoprotein cholesterol (LDL-C), low serum high-density lipoprotein cholesterol (HDL-C), elevated triglycerides, obesity, the presence and levels of C-Reactive Proteins, diabetes mellitus, and advancing age. The quantitative relationship between these risk factors and CHD risk has been elucidated by The Framingham Heart Study and other studies. These studies show that these major risk factors are additive in predictive power. Accordingly the total risk of the person can be estimated by the summing of the risk imparted by each of the major risk factors. Other factors are also associated with increased risk for CHD, for example: (a) Predisposing Risk Factors such as obesity, abdominal obesity, physical inactivity, family history of premature coronary heart disease, ethnic characteristics, psychosocial factors; and (b) Conditional Risk Factors, such as elevated serum triglycerides, small LDL particles, elevated serum homocysteine, elevated serum Lipoprotein (a), prothrombotic factors (e.g. Fibrinogen), and inflammatory markers (e.g. C-Reactive protein).

These risk factors are generally categorized into two types: Predisposing risk factors and Conditional risk factors. Conditional risk factors are associated with increased risk for CHD, although their causative, independent, and quantitative contributions to CHD have not been well documented. Predisposing risk factors are those that worsen the independent risk factors. Two of them—obesity and physical inactivity—are designated major risk factors by the American Heart Association because abdominal obesity is an indicator of insulin resistance. Conditional risk factors are those that have been correlated with CHD risk, but their quantitative relationship to major coronary events remains to be defined adequately in large prospective studies. The predisposing risk factors contribute to the development of the causal and conditional risk factors.

Accordingly, medical therapy for prevention or mitigation of coronary heart disease, stroke, cardiovascular disease, diabetic sequelae, or the reoccurrence of each disease or malady thereof preferably is customized based on the predictive measures disclosed in related US Patent application Ser. No. 11/348,786 and herein.

### Compliance—A Behavioral Phenomenon

Compliance is commonly understood and defined as “the extent to which the patient's behavior (in terms of taking medication, following diets, or executing other lifestyle changes) coincides with medical recommendations.” Compliance is sometimes defined as patients doing what health professionals want them to do. Compliance with prescribed therapeutic regimens has been a documented concern to health professionals since the time of Hippocrates. Patient compliance with medical regimens is a behavioral problem of interest because it affects the patient's health. If the therapeutic regimen is to be effective, the patient must comply with that regimen. No regimen of medication, diet, or behavioral change will benefit the patient who does not follow it.

The role of compliance with medical regimens as a predictor of health outcomes in chronic diseases conditions such as cerebrovascular disorders and diabetes has been the intense focus of recent research. Increasing patient compliance with treatment regimens may decrease hospitalizations and mortality in patient populations as well as improve quality of life (QOL).

Variations in compliance rates have been found to range between 10%-85% depending on the population, the definition of compliance used and the medical regimen studied. E.g., the Pitney-Bowes Compliance Study. While findings have varied, poor compliance with prescribed therapy has been identified in the literature as an issue that encompasses serious problems. Poor compliance has direct negative correlations for the health of the patient, effective use of resources and assessments of the clinical efficacy of the treatment.

Recent noncompliance rates for general health-seeking behaviors and lifestyle modifications are set forth below. The results show low general health-seeking behavior. Not only do patients fail to seek medical attention, they also most likely will not stay in care or comply with follow up appointments over 50% of the time.

TABLE 1
Non-Compliance Behavior Table

| TASKS | RATES |
|---|---|
| Community Screening | 35%-90% |
| Referral After Screening | 50%-65% |
| Staying in Care | 31%-66% |
| Follow-up Appointments | 16%-84% |
| Medications | 31%-58% |
| e.g.: Statin Compliance: | |
| After 3 Months | 60% |
| After 6 Months | 43% |
| After 60 Months | 26% |
| Diet | 13%-76% |
| Physical Activity | 40%-50% |
| Smoking Cessation | 71%-96% |

Even when appropriate treatments are offered, patients do not always adhere to the prescribed treatment regimens. Fourteen to 21% of patients never fill their original prescription. 30%-50% of patients ignore or otherwise compromise their medication instructions.

Compliance rates have been examined for heart failure patients. The results are summarized below. Findings showed that a majority of patients failed to recall elements of potentially important medical advice. Despite some differences in compliance rates in circumstances in which patients did recall medical advice, those that did recall the advice did not always comply with the advice recalled.

TABLE 2
Compliance of Patients with Heart Disease

| | Recalled MD | Noncompliance | Did Not Recall |
|---|---|---|---|
| TASKS | Advice % Total | Rate (%) | Advice |
| Medications | 97.9 | 8.7 | 66.7 |
| Diet (Low Salt) | 83.6 | 23.6 | 55.8 |
| Physical Activity | 70.8 | 76.4 | 84.5 |
| Smoking Cessation | 76.3 | 90.4 | 60.0 |
| Alcohol Use | 42.1 | 60.0 | 81.8 |

In another study involving African American patients with heart conditions measuring the relationship between medication and dietary compliance with hospital readmissions or heart failure HF decompensation, noncompliance was the leading cause for heart failure decompensation, accounting for 43% of hospital admissions. Non-compliance with medication and diet was as high as 64% and 22%, respectively.

Causative factors were identified in 85.5% of the patients in a further study of German heart failure patients. Non-compliance with the medication regimen was the most common identified factor causing heart failure decompensation in 41.9% of cases. Noncompliance with drugs was found in 23% of patients.

A survey of U.S. residents reported that 42% of hypercholesterolemic patients were aware of their condition, although only 4% were adequately treated and controlled.

High Blood Pressure (HBP) is among the most prevalent and important risk factors for cardiovascular, cerebrovascular, and renal disease. Effective care and control of HBP cannot be achieved without compliance to recommended treatment regimens. Estimates of controlled Blood Pressure (BP) among identified HBP patients typically range from 20%-30% in the U.S., in large part, because only one half of the individuals diagnosed with hypertension are in treatment and one half of these are not receiving treatment adequate to control BP.

In another critical review, it was found that noncompliance rates with prescribed therapeutic regimen range from 30%-60%, and at least 50% of patients for whom drugs are prescribed fail to receive full benefit through inadequate compliance. The high noncompliance rates in HBP treatment have multiple implications at the individual and societal levels. These rates jeopardize patients' health and well being, result in suboptimal health outcomes, lead to inefficient use of health resources, and incur costly treatment for the complications of untreated or inadequately treated HBP. In spite of the role played by compliance and the control of HBP, clinicians are not routinely assessing patients' compliance level and patients rarely volunteer this information to their clinician.

The current therapy practice involves prescribing multiple pills for a patient to treat multiple health conditions. Medication compliance for multiple pills is poor. Further, many people forget to take or become confused as to which pills are to be taken at certain times on certain days. The longer the timespan following the doctor's appointment, the greater a failure of medication compliance by the patient will likely occur.

### SUMMARY OF THE INVENTION

Embodiments of the present invention relate to a primary care and/or a secondary care clinical protocol and a therapeutic medical treatment regimen. An aspect of an embodiment of the present invention comprises of a diagnostic criteria, an evaluation of a patient's total risk for Heart Attack, Stroke, and manifestation of diabetic sequelae, a treatment regimen that relates to a selection system enabling the physician or other health care provider to select one of a plurality of one-a-day combinatory drug therapeutic regiments, and a dispensing system for dispensing the selected regiment.

One such selection system includes a printed substrate having at least three parts and each subpart having a sub plurality of indicia representing the one-a-day drug doses and/or treatment regimen formulations and means for moving the sub-parts with respect to each other such as flip charts or pullout plates. In an embodiment, the plurality of indicia are arranged in matrices. In an embodiment, the matrices of data (treatment regimens) are color coded. In an embodiment, the matrices of data are provided to enable the physician to titrate dosages over weekly, monthly or quarterly periods of time based upon the condition of the patient. In an embodiment, a primary matrix has four sub-parts which list treatment regimens (or representative indicia). Further matrices can show additional treatment regimens. Therefore, the selection system enables the physician to pick one selected treatment regimen from the listed formulations, adjust dosages over time (titration of medication), and enables the physician to use the matrices as an educational tool to motivate the patient. The patient sees his or her dosages drop over time by viewing the matrix.

In another embodiment, the selection system comprises an information processing system. The information processing system enables the physician or healthcare provider to select one of the treatment regimens employing a computer (or other electronic device) with a memory, display, and operator input controls. The display shows, upon respective operator inputs, pluralities of treatment regimens and an output generator shows a selected treatment regimen from one of the pluralities of treatment regimens based upon the operators's selection. The information processing system may generate a printed version (script) showing indicia of the selected treatment regimen and may be electronically coupled to a dispensing system which dispenses the selected treatment regimen ordered by the physician.

The dispensing system includes respective storage containers for each formulation of the treatment regimen, each treatment regimen and each respective storage container having a unique formulation of commonly prescribed dosages of widely use medicaments. A treatment regimen dispensing interface accepts a data input from an operator or an electronic data transmission permitting selection of the treatment regimen and dispensing from the corresponding container, the selected treatment regimen. In an embodiment, dispensing control interfaces use an operator confirmation feedback before the pills are finalized for dispensing.

In another aspect of an embodiment of the present invention, a method of treating a patient with a one-a-day, orally administered, treatment regimen is provided, including selecting one of the plurality of treatment regimens and titrating the dosages supplied to the patient after weekly, monthly or quarterly periods of time. Some embodiments of the present invention facilitate the selection of a treatment regimen and/or the change in dosages or titration of dosages of a treatment regimen over time.

In alternative embodiments, rather than combining the customized combination of medications into a single one-a-day treatment regimen, the present invention also relates to a packaging system enabling the physician or other healthcare provider to deliver the medications in a plurality of treatment regimens in a Multi Unit dose (one-a-day or, if necessary, more than once a day) packaging combination to increase compliance with medical therapy. In an embodiment, the Multi Unit Dose Package (“MUDP”) contains one dosage of medicaments prescribed in the treatment regimen. In some embodiments, the MUDP is a pouch containing the medicaments. In embodiments, the MUDP of the subject invention comprises a container that has multiple indented “pie-shaped” pockets. In a particular embodiment, each pie-shaped pocket is separated from the others by plastic packaging material of the MUDP. The packaging system may contain multiple medicaments in the pie-shaped arrangement that may appear as one, preferably multicolored, object when packaged. In embodiments, each medication is provided in a different color. Each treatment regimen of the plurality is selected based on the determined customary therapeutic needs of the individual under treatment. In an embodiment, the selection system and combination therapy matrices of data enable the physician to titrate dosages of each pharmaceutical therapy.

The present invention also relates to the methods of delivery for the treatment regimen Multi Unit Dose Packages over weekly, monthly, quarterly, or longer periods of time based upon the condition of the patient. The use of a MUDP for the individual's treatment regimen with various commonly prescribed dosages of preventive medications may enhance compliance.

The present invention provides packaging for a plurality of pills into a MUDP corresponding to the selection system and the combination treatment matrices mentioned above used in treating a patient for hypertension, hypercholesterolemia, hypertriglyceridemia, anti-platelet aggregation, and related health issues over a weekly, monthly, quarterly, or longer period of time. Ideally, the MUDPs are delivered directly to the patient from the pharmacy. In an embodiment, Multi Unit Dose packaging is provided for complete primary and/or secondary treatment on one strip packaging dispensing system for direct shipment to the patient from a location remote from the patient.

The present invention can further greatly improve patient compliance by providing the Multi Unit Dose Packaging over a period of time (e.g. week, month, quarter, or longer) and having the physician titrate the dosages (adjust the dosages of each effective therapeutic ingredient) over the period of time based upon the condition of the patient. If necessary, the combination treatment pack may provide alternative dosages for treatment at more than one time per day. The MUDP may thus be taken once, two, three, four, or more times per day. The MUDP operates to greatly improve patient compliance with drug treatment regimens and treatment end point elevation to healthier states and potentially reduction for Heart Attack and Stroke to absolute minimums.

An embodiment of the present invention directs the management of the risk factors that cause Cardiovascular and Cerebrovascular accidents. The treatment regimens may include any existing and/or new Drug(s) or Drug Compound that would control the risk factors that would cause Cardiovascular and/or Cerebrovascular accidents. The treatment regimens can also include tests or other activities for the patient (e.g., diet and exercise) in addition to or instead of the prescribed medicaments.

In another aspect of an embodiment of the subject invention, a dispensing device provided capable of detecting when a MUDP or a medicament is used. Thus, the dispensing device can facilitate tracking of the patient's compliance with a treatment regimen. In a further embodiment, a communication device is also incorporated into the dispensing device capable of communicating such information to a computer on a network. Thus, the tracking information can be evaluated and communicated accordingly.

In yet another aspect of an embodiment of the subject invention, a Disease Management System is provided. The Disease Management System can include: a Diagnostic Module, which provides access to patient information and scientific guidelines for patient treatment; a Diagnostic Interpretive Module, which provides tools to evaluate risk of particular diseases or conditions based on patient information and an evaluative methodology; a Prescriptive Module, which is used to recommend, select, and/or evaluate one or more treatment regimens based on patient information and guidelines; a Dispensing Module, which evaluates a patient's compliance with a treatment regimen; and/or a Feedback and Patient Management Module, which gathers compliance information and evaluates efficacy of a treatment regimen for a patient. In embodiments of the subject invention, some or all of the modules described can communicate to manage a disease, medical condition, and/or health problem in a patient.

Although the treatment of CHD, diabetes, and other conditions and their risk factors are described in detail herein, aspects of the present invention can be applied to other diseases, medical conditions, or health problems.

### BRIEF DESCRIPTION OF DRAWINGS

The file of this patent contains at least one drawing executed in color. Copies of this patent with color drawings will be provided by the Patent and Trademark Office upon request and payment of the fee.

In order that a more precise understanding of the above recited invention be obtained, a more particular description of the invention briefly described above will be rendered by reference to specific embodiments thereof that are illustrated in the appended drawings. Understanding that these drawings depict only example embodiments of the invention and are not therefore to be considered as limiting in scope, the invention will be described and explained with additional specificity and detail through the use of the accompanying drawings in which:

FIG. 1 diagrammatically illustrates an information processing system for selecting a treatment regimen from a plurality of treatment regimens in accordance with an embodiment of the subject invention;

FIG. 2 diagrammatically illustrates a dispensing system for a plurality of medicaments in accordance with an embodiment of the subject invention;

FIG. 3 diagrammatically illustrates a dispensing system for a plurality of medicaments in accordance with another embodiment of the subject invention;

FIG. 4 diagrammatically illustrates a dispensing system for a plurality of medicaments in accordance with yet another embodiment of the subject invention;

FIGS. 5A-5E diagrammatically illustrate a system for selecting a treatment regimen from a plurality of treatment regimens in accordance with an embodiment of the subject invention;

FIGS. 6A-6C diagrammatically illustrate a system for selecting a treatment regimen from a plurality of treatment regimens in accordance with another embodiment of the subject invention;

FIG. 7 diagrammatically illustrates a system for selecting a treatment regimen from a plurality of treatment regimens in accordance with yet another embodiment of the subject invention;

FIGS. 8A-8C illustrate various apparatuses for packaging multiple medicaments for convenient dosing according to embodiments of the subject invention;

FIG. 9 diagrammatically illustrates a method for diagnosing disease in a patient in selecting a treatment regimen in accordance with an embodiment of the subject invention;

FIG. 10 diagrammatically illustrates a method for delivering a medicament to a patient in accordance with an embodiment of the subject invention;

FIG. 11 illustrates example apparatuses for packaging one or more medicaments for delivery to a patient in accordance with an embodiment of the subject invention;

FIG. 12 diagrammatically illustrates a Disease Management System in accordance with an embodiment of the subject invention;

FIG. 13 illustrates an apparatus for dispensing medicaments in accordance with an embodiment of the subject invention;

FIG. 14 illustrates an interface to a Diagnostic Module in accordance with an embodiment of the subject invention;

FIG. 15 illustrates an interface to a Disease Management System in accordance with an embodiment of the subject invention;

FIG. 16 illustrates an interface to a Diagnostic Module in accordance with an embodiment of the subject invention;

FIG. 17 illustrates an interface to a Diagnostic Module in accordance with an embodiment of the subject invention;

FIG. 18 illustrates an interface to a Diagnostic Module in accordance with an embodiment of the subject invention;

FIG. 19 illustrates an interface to a Diagnostic Module in accordance with an embodiment of the subject invention;

FIG. 20 illustrates an interface to a Feedback and Patient Management Module in accordance with an embodiment of the subject invention;

FIG. 21 illustrates an interface to a Feedback and Patient Management Module in accordance with an embodiment of the subject invention;

FIG. 22 illustrates an interface to a Diagnostic Interpretive Module in accordance with an embodiment of the subject invention; and

FIGS. 23A-C illustrate an interface to a Prescriptive Module in accordance with an embodiment of the subject invention.

### DETAILED DISCLOSURE

The present invention relates to a selection system for selecting one of a plurality of one-a-day combination medical treatment regimen, or alternatively Multi Unit Dose Package(s), for treatment of hypertension, hypercholesterolemia, hypertriglyceridemia and antiplatelet treatment, an information processing system therefore, a dispensing system for dispensing the selected one-a-day combination medical treatment regimen or MUDP from said plurality of one-a-day combination medical treatment regimen and MUDP(s), and a method of treatment using said one-a-day combination medical treatment regimen or MUDP, and a follow-up or feedback system to monitor the patients state of health and compliance.

In certain aspects, the present invention relates to a packaging system for the delivery of a medical treatment regimen for hypertension, hypercholesterolemia, hypertriglyceridemia, anti-platelet aggregative treatment, treatment of type 2 diabetes and/or reduction and postponement of diabetic sequelae, among other conditions or health issues. In an embodiment, the packaging system utilizes the drug combination matrices and a clinical protocol to provide a MUDP to increase patient compliance with drug treatment. In an embodiment, the combination matrices and clinical protocol are determined from the risk factor studies as detailed below and incorporated from U.S. patent application Ser. No. 11/348,786. Therefore, customized treatment protocols have been determined to optimize patient response, disease management, and evidenced based treatment medical regimen. In addition to the treatment protocol, the present invention maximizes treatment efficacy by improving aspects of drug delivery to patients to counteract detrimental behavioral issues, namely patient compliance with drug treatment regimens. In an embodiment, MUDPs are provided in a strip format for variable therapeutic periods and time frames.

In an embodiment of the invention, the Multi Unit Dose Packaging system comprises an individual pouch to contain the patient's determined prescribed therapy. In embodiments, the pouch contains a plurality of pocketed or indented compartments to hold therapeutic regimen or medication. The pouch commonly contains a range of two to six, and occasionally even more, separate and distinct pockets or indents to hold the therapeutic regimen or medication. The distinct pockets or indents can be separated within the pouch by a thin partition. Thus, each therapeutic regimen or medication can be separated from the other constituents of the pouch. The pouch thus comprises a therapeutic regimen container, and serves as a Multi Unit Dose Package, to simplify the patient's treatment protocol.

In another embodiment of the invention, the MUDPs are then packaged into MUDP supply boxes containing a plurality of MUDPs. In an embodiment, the MUDPs can be packaged into 7-day, 30-day, 90-day, or even longer supply boxes, customized for the patient. In an embodiment, the supply boxes contain one MUDP corresponding to each day of the treatment indicated on the supply box. The physician can therefore direct the pharmacy to deliver direct to the patient the drug treatment customized for each individual patient in both a condensed MUDP and long-term package to simplify patient compliance.

In yet other embodiments, each MUDP can represent one combination dosage of multiple prescribed daily drug treatments. The MUDP can be taken one, two, three, four, or more times per day as prescribed. Accordingly, the long-term MUDP supply boxes reflect the increase in daily drug treatment with a corresponding increase in MUDPs delivered by the supply boxes.

In another aspect of an embodiment of the subject invention, a dispensing device is incorporated into the supply box capable of detecting when a MUDP or a medicament is removed from the supply box or when such an event has not occurred within a given period of time. Thus, the dispensing device can facilitate tracking of the patient's compliance with a treatment regimen. In a further embodiment, a communication device is also incorporated into the dispensing device capable of communicating such information to a computer on a network. Thus, the tracking information can be evaluated and communicated accordingly.

In yet another aspect of an embodiment of the subject invention, a Disease Management System is provided. The Disease Management System can include: a Diagnostic Module, which provides access to patient information and scientific guidelines for patient treatment; a Diagnostic Interpretive Module, which provides tools to evaluate risk of particular diseases or conditions based on patient information and an evaluative methodology; a Prescriptive Module, which is used to recommend, select, and/or evaluate one or more treatment regimens based on patient information and guidelines; a Dispensing Module, which evaluates a patient's compliance with a treatment regimen; and/or a Feedback and Patient Management Module, which gathers compliance information and evaluates efficacy of a treatment regimen for a patient. In embodiments of the subject invention, some or all of the modules described can communicate to manage a disease, medical condition, and/or health problem in a patient.

The subject matter of the present invention is described with specificity to meet statutory requirements. But this description is not intended to limit the scope of this patent. Rather, the inventors have contemplated that the claimed subject matter might also be embodied in other ways, to include different steps or combinations of steps similar to those described in this document, in conjunction with other present or future technologies.

Aspects of the invention can be described in the general context of computer-executable instructions, such as program modules, being executed by a computer. Generally, program modules include routines, programs, objects, components, data structures, etc., that perform particular tasks or implement particular abstract data types. Moreover, those skilled in the art will appreciate that the invention can be practiced with a variety of computer-system configurations, including multiprocessor systems, microprocessor-based or programmable-consumer electronics, minicomputers, mainframe computers, and the like. Any number of computer-systems and computer networks are acceptable for use with the present invention.

Specific hardware devices, programming languages, components, processes, protocols, formats, and numerous other details including operating environments and the like are set forth to provide a thorough understanding of the present invention. In other instances, structures, devices, and processes are shown in block-diagram form, rather than in detail, to avoid obscuring the present invention. But an ordinary-skilled artisan would understand that the present invention can be practiced without these specific details. Computer systems, servers, work stations, and other machines can be connected to one another across a communication medium including, for example, a network or networks.

As one skilled in the art will appreciate, embodiments of the present invention can be embodied as, among other things: a method, system, or computer-program product. Accordingly, the embodiments can take the form of a hardware embodiment, a software embodiment, or an embodiment combining software and hardware. In one embodiment, the present invention takes the form of a computer-program product that includes computer-useable instructions embodied on one or more computer-readable media.

Computer-readable media include both volatile and nonvolatile media, removable and nonremovable media, and contemplate media readable by a database, a switch, and various other network devices. By way of example, and not limitation, computer-readable media comprise media implemented in any method or technology for storing information. Examples of stored information include computer-useable instructions, data structures, program modules, and other data representations. Media examples include, but are not limited to, information-delivery media, RAM, ROM, EEPROM, flash memory or other memory technology, CD-ROM, digital versatile discs (DVD), holographic media or other optical disc storage, magnetic cassettes, magnetic tape, magnetic disk storage, and other magnetic storage devices. These technologies can store data momentarily, temporarily, or permanently.

The invention can be practiced in distributed-computing environments where tasks are performed by remote-processing devices that are linked through a communications network. In a distributed-computing environment, program modules can be located in both local and remote computer-storage media including memory storage devices. The computer-useable instructions form an interface to allow a computer to react according to a source of input. The instructions cooperate with other code segments or modules to initiate a variety of tasks in response to data received in conjunction with the source of the received data.

The present invention can be practiced in a network environment such as a communications network. Such networks are widely used to connect various types of network elements, such as routers, servers, gateways, and so forth. Further, the invention can be practiced in a multi-network environment having various, connected public and/or private networks.

Communication between network elements can be wireless or wireline (wired). As will be appreciated by those skilled in the art, communication networks can take several different forms and can use several different communication protocols. And the present invention is not limited by the forms and communication protocols described herein.

The invention is described more fully hereinafter with reference to the accompanying drawings, in which embodiments of the invention are shown. This invention can, however, be embodied in many different forms and should not be construed as limited to the embodiments set forth herein. Rather, these embodiments are provided to fully enable those of ordinary skill in the art to embody and practice the invention.

In an embodiment, the treatment protocols set forth herein and accomplished by use of the MUDP and/or Disease Management System are aimed at reducing the incidence of cerebrovascular and cardiovascular diseases, and diabetic sequelae by primary prevention approaches. In an embodiment, the therapeutic modality utilized is that of secondary prevention. In an embodiment, preventive efforts target each major therapeutically modifiable risk factor. In an embodiment, the treatment protocol assesses global risk based on the summation of risk factors and utilizes them clinically by (a) identifying and measuring patients at risk and (b) managing risk factors. In an embodiment, the protocol identifies and measures patients at risk—patients at high risk and patients at low risk—for immediate attention and intervention of the major modifiable risk factors by secondary treatment protocols. In an embodiment, risk factor management entails: (a) Patient enlightenment and motivation, that is, motivate patients to join and adhere to traditional risk reduction therapies ranging in spectrum from diet modification, lifestyle changes to medical therapy; and (b) Modification of the intensity of risk-reduction therapy based upon the global risk estimate, such as stringent diet, exercise, and titration of medication to maintain the risk factors within the parameters of a low risk state.

In an embodiment, the first step in the implementation of the MUDP combination therapeutic regimen treatment is to identify the risk of the patient to CHD, cerebrovascular disease and diabetic sequelae. There are several clinical protocols which may be employed to determine the risk to the patient. The Framingham Heart Study or Framingham Report defined Low Risk as the risk for CHD at any age that is conferred by any combination of all the following parameters: blood pressure<120/<80 mm Hg., total cholesterol 160 to 199 mg./dL. (or LDL-C 100 to 129 mg/dL) for men and 55 mg/dL for women in a non-smoking person with no diabetes. The Framingham study defines a low-risk state as: serum total cholesterol 160 to 199 mg/dL; LDL-C 100 to 129 mg/dL; HDL-C 45 mg/dL. in men and 55 mg/dL in women, blood pressure<120 mm Hg systolic and <80 mm Hg diastolic, nonsmoker; no diabetes mellitus. Table 3 provides one clinical protocol for accessing hypertension in a patient.

TABLE 3
Hypertension can be rated as follows:

| | Systolic | | Diastolic | |
|---|---|---|---|---|
| Category | (mmHg) | | Result | |
| Normal | 115 or | And | 75 or | Good for you |
| | lower | | lower | |
| Prehyper- | 115-139 | Or | 75-89 | Your blood pressure could be |
| tension | | | | a problem. Make changes in |
| | | | | what you eat and drink, be |
| | | | | physically active and lose |
| | | | | extra weight. If you also have |
| | | | | diabetes, see your physician. |
| Hyper- | 140-159 | Or | 90-99 | You have high blood pressure. |
| tension | | | | Ask your doctor or nurse how |
| Stage 1 | | | | to control it. |
| Hyper- | 160 or | Or | 100 or | You have high blood pressure. |
| tension | higher | | higher | |
| Stage 2 | | | | |

Government agencies have recommended the following treatment for hypertension: Stage 1 hypertension: thiazide-type diuretics, ACE Inhibitors, Beta Blockers, ACEI, ARB, BB, CCB; Stage 2 hypertension: 2 drug combination—thiazide-type diuretics and ACEI, ARB, BB or CCB. After this treatment, if the patient is not at blood pressure or BP goal, optimize dosages or add additional drugs until goal BP is reached.

Absolute risk is defined as the probability of developing CHD over a given period of time. A recent Framingham report specifies absolute risk for CHD over the next 10 years. The relative risk is the ratio of the absolute risk of the given patient (or group) to that of low-risk group. Literally, the term relative risk represents the ratio of the incidence in the exposed population divided by the incidence in the unexposed persons. The denominator of the ratio can be either the average risk of the entire population or the risk of a group devoid of risk factors. Both the absolute and relative risk is derived from the recently published risk score sheets.

One methodology for risk estimation builds upon the Framingham study and assigns points for indications of various risk factors. For example, risk factors can include age (in gradations from in 5 year blocks from age 30 and points from −1 to 7 up to age 74), LDL cholesterol, HDL cholesterol, total cholesterol, blood pressure (BP) (systolic, diastolic, either, or both), diabetes (male: Y or N, 0 or 2 points or female: Y or N, 0 or 4 points, defined as a fasting plasma glucose level>126 md/dL), smoker (Y or N, 0 or 2 points, defined any smoking in the past month). Other risk factors can be included, for example hyperlipidemia and obesity, which can be defined by Body Mass Index (BMI) or another method known in the art, among other factors. In addition, the number of points assigned to various levels of indications of risk factors may also vary. In a further embodiment, the points from the various risk factors are totaled to provide a point total that is correlated with an estimated risk based on those factors. Table 4, below, provides one example of a scoring protocol referred to as the Framingham Scoring System. Other protocols or variations on this protocol will be known to those skilled in the art and can be used with the subject invention.

TABLE 4
Global Risk Assessment Scoring Chart

| | | |
|---|---|---|
| Risk | Male | Female |

Age

| | | |
|---|---|---|
| <35 | −1 | −9 |
| 35-39 | 0 | −4 |
| 40-44 | 1 | 0 |
| 45-49 | 2 | 3 |
| 50-54 | 3 | 6 |
| 55-59 | 4 | 7 |
| 60-64 | 5 | 8 |
| 65-69 | 6 | 8 |
| >69 | 7 | 8 |

Total Cholesterol (mg/dl)

| | | |
|---|---|---|
| <160 | −3 | −2 |
| 160-199 | 0 | 0 |
| 200-239 | 1 | 1 |
| 240-279 | 2 | 2 |
| >279 | 3 | 3 |

HDL Cholesterol (mg/dl)

| | | |
|---|---|---|
| <35 | 2 | 5 |
| 35-44 | 1 | 2 |
| 45-49 | 0 | 1 |
| 50-59 | 0 | 0 |
| >59 | −2 | −3 |

Systolic Blood Pressure (mmHg)

| | | |
|---|---|---|
| <120 | 0 | −3 |
| 120-129 | 0 | 0 |
| 130-139 | 1 | 1 |
| 140-159 | 2 | 2 |
| >159 | 3 | 3 |

Diastolic Blood Pressure (mmHg)

| | | |
|---|---|---|
| <85 | 0 | 0 |
| 85-89 | 1 | 1 |
| 90-99 | 2 | 2 |
| >99 | 3 | 3 |

Diabetes

| | | |
|---|---|---|
| No | 0 | 0 |
| Yes | 2 | 4 |

Smoker

| | | |
|---|---|---|
| No | 0 | 0 |
| Yes | 2 | 2 |

According to this protocol, points for age, total cholesterol, HDL cholesterol, blood pressure, diabetes, are assessed and the points are totaled to obtain a Framingham Score. In a variation on this protocol, LDL cholesterol, and/or the ratio between LDL cholesterol and HDL cholesterol, can be assessed in addition to or instead of total cholesterol. In another variation, only systolic pressure is assessed, or both systolic and diastolic pressure are assessed but only the category with the greatest point result is counted toward the total. Next, a 10 year CHD risk projection can be correlated with the total points. Table 5 provides CHD Risk estimates based on scores developed from the protocol of Table 4 (the Framingham Scoring System).

TABLE 5
CHD Risk Table (determined CHD risk from Framingham Score)

| Point Total | 10 Yr CHD Risk |
|---|---|
| <−3 | 1% |
| −2 | 2% |
| −1 | 2% |
| 0 | 3% |
| 1 | 4% |
| 2 | 4% |
| 3 | 6% |
| 4 | 7% |
| 5 | 9% |
| 6 | 11% |
| 7 | 14% |
| 8 | 18% |
| 9 | 22% |
| 10 | 27% |
| 11 | 33% |
| 12 | 40% |
| 13 | 47% |
| >14 | >58% |

The individual's CHD risk can be compared to his or her population as follows.

TABLE 6
CHD Comparison to Others Table (compared
to man of the same age)

| | Average 10 Yr | Low 10 Yr |
|---|---|---|
| Age (years) | CHD Risk | CHD Risk |
| 30-34 | 3% | 2% |
| 35-39 | 5% | 3% |
| 40-44 | 7% | 4% |
| 45-49 | 11% | 4% |
| 50-54 | 14% | 6% |
| 55-59 | 16% | 7% |
| 60-64 | 21% | 9% |
| 65-69 | 25% | 11% |
| 70-74 | 30% | 14% |

Being overweight or obese increases the risk of developing high blood pressure and type 2 diabetes. Blood pressure (BP) rises as body weight increases. Studies have shown that losing even 10 pounds can lower the blood pressure and losing weight has the biggest effect on those who are overweight and already have hypertension. The two measures used to determine factors of overweight or obesity are body mass index, or BMI, and waist circumference. BMI is a measure of weight relative to height. It gives an approximation of total body fat. By motivating the patient to lose weight, his or her blood pressure improves.

Table 7 provides relative and absolute risk estimates for CHD in men as determined based on Framingham Score. The relative risk estimates for each age range are compared with baseline risk conferred by age alone (in the absence of other major risk factors). Relative risk is graded to include below average, average and moderately above average and high-risk categories. Distinctions in relative risk are arbitrary. Average risk refers to that observed in the Framingham population. Absolute risk estimates are given in the two right hand columns. Absolute risk is expressed as a percentage likelihood of developing CHD per decade. Total CHD risk equates to all forms of clinical CHD, whereas hard CHD includes clinical evidence of myocardial infarction and coronary death. Hard CHD estimates are approximated from the published Framingham Data.

TABLE 7
Risk Assessment Table.

| | Age | | | | | | | | | | |
|---|---|---|---|---|---|---|---|---|---|---|---|
| | 30-34 | 35-39 | 40-44 | 45-49 | 50-54 | 55-59 | 60-64 | 65-69 | 70-74 | Absolute | Absolute |
| | Low Risk Level | | | | | | | | | Risk | Risk |
| Points | (2%) | (3%) | (4%) | (5%) | (6%) | (7%) | (8%) | (10%) | (13%) | Total CHD | Hard CHD |
| 0 | 1.0 (1) | | | | | | | | | 2% | 2% |
| 1 | 1.5 (2) | 1.0 | 1.0 | | | | | | | 3% | 2% |
| 2 | 2.0 (3) | 1.3 (1) | 1.3 | 1.0 | | | | | | 4% | 3% |
| 3 | 2.5 (3) | 1.7 (2) | 1.7 (1) | 1.3 | 1.0 | | | | | 5% | 4% |
| 4 | 3.5 (3) | 2.3 (3) | 2.3 (2) | 1.8 | 1.4 | 1.0 | | | | 7% | 5% |
| 5 | 4.0 (4) | 2.6 (3) | 2.6 (2) | 2.0 (1) | 1.6 | 1.1 | 1.0 | | | 8% | 6% |
| 6 | 5.0 | 3.3 (3) | 3.3 (3) | 2.5 (2) | 2.0 (1) | 1.4 | 1.3 | 1.0 | | 10% | 7% |
| 7 | 6.5 | 4.3 (4) | 4.3 (4) | 3.3 (3) | 2.6 (2) | 1.9 (1) | 1.6 | 1.3 | 1.0 | 13% | 9% |
| 8 | 8.0 | 5.3 | 5.3 | 4.0 (4) | 3.2 (3) | 2.3 (2) | 2.0 (1) | 1.6 | 1.2 | 16% | 13% |
| 9 | 10.0 | 6.7 | 6.7 | 5.0 | 4.0 (4) | 2.9 (3) | 2.5 (2) | 2.0 (1) | 1.5 | 20% | 16% |
| 10 | 12.5 | 8.3 | 8.3 | 6.3 | 5.0 | 3.6 (4) | 3.1 (3) | 2.5 (2) | 1.9 (1) | 25% | 20% |
| 11 | 15.5 | 10.3 | 10.3 | 7.8 | 6.1 | 4.4 | 3.9 (4) | 3.1 (3) | 2.3 (2) | 31% | 25% |
| 12 | 18.5 | 12.3 | 12.3 | 9.3 | 7.4 | 5.2 | 4.6 | 3.7 (4) | 2.8 (3) | 37% | 30% |
| 12 | 22.5 | 15.0 | 15.0 | 11.3 | 9.0 | 6.4 | 5.6 | 4.5 | 5.3 (4) | 45% | 35% |
| >14 | 26.5 | >17.7 | >17.7 | >13.3 | >10.6 | >7.6 | >6.6 | >5.3 | >4.1 | >53% | >45% |

Boundary markers for risk categories denoted in ( ):
(1) = Below Average Risk;
(2) = Average Risk;
(3) = Moderately Above Average Risk;
(4) = High Risk

“Low Risk Level” provides the 10-year absolute risk for total CHD end points for persons in the age group with good blood pressure (<120/<80 mmHg.), total cholesterol (160-199 mm/dL.), HDL-C mg/dL., nonsmoker, and no diabetes. Points are the number of points accumulated from the Global Risk Assessment Scoring Chart provided above. The absolute risk columns represent the 10-year absolute risk for total CHD and hard CHD end points respectively and are estimated from Framingham Data corresponding to Framingham Score.

The Framingham Scoring System takes into account gradations in risk factors when estimating absolute risk. The scoring does not adequately account for severe abnormalities or risk factors, e.g. severe hypercholesterolemia, severe hypertension, uncontrolled diabetes, or cigarette smoking. In these cases, Framingham scores can underestimate absolute risk. This underestimation is particularly evident when only one risk factor is present. Thus, heavy smoking or severe hypercholesterolemia can lead to premature CHD even when the summed score for the absolute risk is not high. Similarly, the many dangers of prolonged, uncontrolled hypertension are well known. Thus, in embodiments, a physician is consulted to exercise subjective clinical acumen to control severe risk factors regardless of absolute short-term risk estimates. The Framingham Scoring System is merely one example of a system known in the art for estimating disease risk. Other systems are known and can be used with the subject invention.

In an embodiment, the protocol for selecting which “Multi Unit Drug Packaging” (MUDP) combination pill treatment or one-a-day treatment regimen formulation should be prescribed to a patient is based upon an initial screening and assessment of a patient. In an embodiment, this includes (1) measurement of serum levels of total cholesterol (or LDL-C) and HDL-C and evaluation of cholesterol disorders requires measurement of LDL-C, which is the primary target of cholesterol lowering therapy; and (2) measurement of blood pressure (regardless of whether the patient is taking antihypertensive drugs. The average of several blood pressure measurements can be used for an accurate determination of the baseline level pursuant the CDC recommended protocols.

Other factors such as the patient's age, ECG or EKG abnormalities, ABI tests, B-mode ultrasound of carotid, aorta and femoral arteries, ultrasound of carotid arterial intima and media thickness are also diagnostic tools that may be employed by the physician to determine which of the many one-a-day treatment regimens should be prescribed in the Multi Unit Drug Packaging System (MUDP) should be prescribed.

A patient's age is an indicator of absolute risk, because it reflects the total burden of atherosclerosis that has accumulated; the probability of suffering a major coronary event (unstable angina or myocardial infarction) is correlated with total plaque burden.

ECG or EKG abnormalities, such as abnormalities in the rest ECG, nonspecific ST-segment changes and left ventricular hypertrophy, also carry predictive power and can improve office-based risk assessment.

Noninvasive tests of atherosclerotic Burde Ankle-brachial blood pressure Index (ABI) is a simple diagnostic test for lower-extremity peripheral arterial disease (PAD). It is simply the ratio of blood pressure measured in the arteries at the foot or ankle (dorsalis pedis and posterior tibialis arteries, measured by a hand-held Doppler probe) to the blood pressure measured by traditional blood pressure cuff in the arm (brachial artery). Among well-trained operators, test-retest reliability is excellent and the validity of the test for =50% stenosis in leg arteries is high (90% sensitivity and 98% specificity). In population studies, patients with low ABI have been found to have a considerably higher prevalence of CVD (history of myocardial infarction, coronary artery bypass graft surgery, stroke or stroke surgery, or other measures of clinical CVD such as angina or congestive heart failure) compared to those with normal ABI. Such data confirm that atherosclerosis is a diffuse (i.e., systemic) disease and that an abnormal ABI test (ratio<0.90) suggests significant atherosclerosis in other vascular beds. At least 3 prospective studies have shown a strong predictive role for the ABI for CVD morbidity and mortality prediction in persons with PAD detected by ABI.

Many asymptomatic persons aged 50 and over will have abnormal ABI values. Follow-up studies have shown that abnormal ABI provides incremental coronary and all-CVD risk assessment information, over and above that provided by traditional risk factors. For example, in one study, an abnormal ABI increased relative risk for CVD mortality by nearly 4-fold over standard CV risk factors.

B-mode ultrasound is a relatively inexpensive and safe technique that visualizes the lumen and walls of selected arteries, including carotid, aorta, and femoral. B-mode ultrasound has been validated for measuring intima-media thickness (IMT). Cross-sectional associations between common carotid artery IMT and CVD risk factors have been demonstrated in several studies. Similarly, common carotid IMT has been associated with prevalent CVD in cross-sectional studies. At least 4 published studies show that carotid IMT measurement predicts the presence of CHD and its clinical sequelae.

Noninvasive measurements of the intima and media of the common and internal carotid arteries made with high-resolution ultrasonography can form a base for risk diagnostic purposes. The incidence of cardiovascular events has been correlated with measurements of carotid-artery intima-media thickness. The relative risk of myocardial infarction or stroke increased with intima-media thickness. The relative risk of myocardial infarction or stroke (adjusted for age and sex) for the quintile with the highest thickness as compared with the lowest quintile was 3.87 (95% confidence interval, 2.72 to 5.51). The association between cardiovascular events and intima-media thickness remained significant after adjustment for traditional risk factors, showing increasing risk for each quintile of combined intima-media thickness, from the second quintile (relative risk, 1.54, 95% confidence interval, 1.04 to 2.28), to the third (relative risk 1.84, 9r % confidence interval, 1.26 to 2.67), fourth (relative risk, 2.01; 95% confidence interval, 1.38 to 2.91) and fifth (relative risk, 3.15; 95% confidence interval, 2.19 to 4.52). The results of separate analyses of myocardial infarction and stroke paralleled those for the combined endpoint. The study showed that increases in the thickness of the intima and media of the carotid artery, as measured noninvasive by ultrasonography, are directly associated with an increased risk of myocardial infarction and stroke in older adults without a history of cardiovascular disease.

Prevention of clinical atherosclerotic sequelae (myocardial infarction, stroke and peripheral vascular disease) can involve modifying reversible risk factors such as systemic hypertension, dyslipidemia, hypertriglyceridemia, tobacco smoking and its consequent elevation of platelet aggregation, and excess body mass index. Studies have demonstrated that atherosclerosis begins at an early age and progresses in an asymptomatic manner over decades.

Patients at high-risk because of multiple risk factors may require intensive modification of risk factors to maximize risk reduction. These guidelines are currently endorsed or supported by various medical organizations and governmental bodies. The reports advocate adjusting the intensity of risk-factor management to the global risk of the patient. In certain reports, overall risk is estimated by adding the categorical risk factors. They do not use a total risk estimate based on summation of risk factors that have been graded according to risk severity. This latter approach is advocated by the Framingham investigators. Framingham reported that some clinicians believe that the summation of graded risk factors provides advantages over the addition of categorical risk factors. The use of graded risk factors has been recommended in risk-management guidelines developed in Europe.

There are several independent factors that can affect the selection and use of the “Multi Unit Drug Packaging” (MUDP) treatment regimen or one-a-day treatment regimen. These are: (a) Diabetes Mellitus (a major risk factor for CHD, both Type I and Type II); (b) Elderly Patients (a prominent feature of the Framingham risk score); (c) Hypertriglyceridemia (elevated serum triglycerides are independent risk factor and elevated triglycerides consequently become a target of therapy independent of LDL lowering); (d) Family History of Premature CHD (imparts an incremental risk at any level of global risk factors); (e) Psychosocial Factors (contribution of personality and socioeconomic factors such as economic standing, evocation, racial background, lifestyle, and personality type, increase CHD risk); and/or (f) Homocysteine (high serum concentration of homocysteine is associated with increased risk for CHD). Not all factors are used in all embodiments of the subject invention. Any subset may be selected or used, or in other embodiments none of these factors are used.

### Treatment of Diabetes with the MUDP Treatment Regimen

In an embodiment, the MUDP treatment regimen can also be used to delay the onset of type 2 diabetes or the progression of diabetic sequelae. The primordial risk factors for CHD are the same as those which cause targeted organ disease, such a type 2 diabetes, that is high blood pressure, cholesterol and triglyceride levels. Patients with type 2 diabetes often have high blood pressure and high cholesterol and are at increased risk of heart attack and stroke. With the MUDP treatment regimen, (a) patients with type 2 diabetes can have a greater medication compliance (b) can be less subject to the “sick patient” syndrome; (c) the cost of treatment should be less due to the reduced cost of medication (rather than take 4-5 medications, a MUDP with 4-5 medications is more cost effective); and/or (d) the total healthcare cost for the patient with type 2 diabetes can be reduced since the risk of heart attack and stroke and subsequent treatment therefore is reduced. Since patients with type 2 diabetes sometimes take several other medicaments each day, a reduction of 4-5 pills and the substitution of a MUDP treatment regimen can increase compliance and improve patient health. The MUDP treatment regimen can also reduce patient error involving taking the wrong pills in the wrong dosages.

### Primary Care Usages and Secondary Care Usage

The MUDP treatment regimen is effective both as a primary care treatment and as a secondary care treatment plan. Primary care is called for before the patient suffers from his or her first heart attack or stroke (prior to diagnosis of the CVD or CHD ailment and type 2 diabetes), or other condition. The risk factor assessment discussed herein enables the physician to employ the MUDP treatment regimen as a primary care treatment plan. The doctor and the patient can see improvements by titration of dosages and with the use of the treatment regimen Matrix discussed below.

Secondary care can also involve the use of the MUDP treatment regimen. After a heart attack or a stroke or onset of another condition, the MUDP treatment regimen may be used to titrate the correct dosages for the patient. After the patient leaves the health care facility, the MUDP treatment regimen is easier to prescribe and its use increases patient compliance. Further, patient memory loss, even on a temporary basis, sometimes reduces medication compliance. The MUDP treatment regimen improves compliance since the entire drug treatment regimen is contained in one easy to use package. In an embodiment, the primary care protocol also prescribes the use of a thrombolytic agent. The FDA has indicated that the thrombolytic agent, in the formulations set forth herein, may be taken 4, 8, 16 hours after a Heart Attack as Secondary care in conventional, conservative therapy. The primary care protocol has also been approved by the FDA in an IND Waiver for the use as a thrombolytic agent in the event of an impending cerebrovascular accident whose etiology is based on a stenosed artery due to the accumulation of plaque. A thrombolytic agent is generally not taken unless the target blood vessel is at least 66% occluded., i.e. Streptokinase or a new generation thrombolytic agent.

### MUDP Treatment Regimen, Clinical Protocol, Risk Factor Management

Having established the absolute risk of the patient to CHD, cerebrovascular disease and diabetic sequelae, the MUDP treatment regimen protocol can address the management of these independent modifiable risk factors.

In an embodiment of the present invention, diagnostic criteria of the Cerebrovascular and Cardiovascular status and diabetic sequelae status are used to establish a process to assure compliance and titration with administration of medication to maintain the risk factors within the parameters of a low risk state, and the administration of thrombolytics when clinically indicated. In an embodiment, upon establishment of absolute risk and selection of the patient for treatment, the patient is treated aggressively as follows. The patient is interviewed in detail with the objective of gleaning information concerning implementation of behavioral and lifestyle modification changes, i.e. diet, exercise and rest. The physician needs to establish a chemotherapeutic regimen prescriptive to the unique needs of the patient. Each modifiable risk factor, such as: hypertension, hypercholesterolemia, hypertriglyceridemia, blood glucose levels (diabetes) and the elevated aggregative potential of platelets caused by smoking, is assessed individually and treated.

Each of the medical compounds in the MUDP treatment regimen targeted at reducing the risk potential of the modifiable risk factors is prescribed by many medical protocols. In an embodiment, to improve the probability of compliance of taking prescribed medication at a specified time of the day, the MUDP treatment regimen protocol requires medication aimed at treating elevated BP (i.e., ACE inhibitor, B-Blocker, Diuretic, etc.), and medication aimed at treating hypercholesterolemia (i.e., A Statin drug compound—LIPITOR, ZOCOR, etc.), Elevated triglycerides (i.e., TRICOR—fenofibrate), type 2 diabetes (i.e. Insulin and/or DIABENESE), and an anti-platelet aggregative medication (i.e., PLA VIX/Aspirin), can be combined in one MUDP treatment regimen. The MUDP treatment regimen can encourage compliance and assist in eliminating the notions of “a sick person.”

In an embodiment, the patient is evaluated at least monthly for the first three months for purposes of titration of dosage and monitoring of side effects for the medications. Titration of the medication is an essential and integral part managing potentially modifiable risk factors. In other embodiments, the patient is evaluated more or less frequently. For example, in an embodiment the patient is evaluated weekly for four to six weeks, or longer. Other useful frequencies can be suggested by one skilled in that are and can be used with the subject invention.

Since the risk assessment can vary for each patient and the patient's dosage for each element in the MUDP treatment regimen can change over time, it is helpful to present to the physician a matrix of available treatment regimens. As noted below, in an embodiment, the matrix is quite long and complex. In an embodiment, the designating indicia S-D-A-T adjacent the mg dosage also greatly assists the physician in selecting one of the treatment regimens. In an embodiment, upon reaching the physician's expectations of maximal management results based upon further visits over time and titration of dosage, office visits are then prescribed by the physician based upon individual patient histories.

An embodiment of the MUDP treatment regimen Matrix (below) is understood by the following example: First, each one-day MUDP Combination Medical Therapeutic Regimen has either aspirin or clopidogrel (a.k.a. PLAVIX) or both and each unique MUDP treatment regimen is designated with an S-D-A-T indicia code such as S10-D25-A10-T160 wherein S=Statin Drugs to lower cholesterol; D=Diuretic—a drug to reduce the water in the patient's body thus lowering his or her blood pressure; A=ACE Inhibitor—a hypertension drug that inhibits substances in the body from producing substances that cause high blood pressure (e.g., the following is a list of some Inhibitors that are available in the United States: captopril (CAPOTEN), benazepril (LOTENSIN), enalapril (VASOTEC), lisinopril (PRINIVIL, ZESTRIL) fosinopril (MONOPRIL), ramipril (ALTACE), perindopril (ACEON), quinapril (ACCUPRIL), moexipril (UNIVASC), and trandolapril (MAVIK)); T=TRICOR—a drug used to lower triglycerides and, to a small degree, cholesterol. TRICOR (fenofibrate tablets), is a lipid regulating agent available as tablets for oral administration; and the addition of one or more diabetic treatment drug compound. Each tablet contains fenofibrate. The chemical name for fenofibrate is 2-[4-(4-chlorobenzoyl)phenoxy]-2-methyl-propanoic acid, 1-methylethyl ester.

The number following the letter is the medicament's strength in milligrams. For example, S10-D25-A10-T160 is equivalent to S10 or 10 mg of a statin; D25 is 25 mg of a diuretic; A10 is 10 mg of an ACE Inhibitor; and T160 is 160 mg of TRICOR. People need various strengths of these widely prescribed drugs to control their risk factors, cholesterol, hypertension, and triglycerides. Rather than giving a person 4 or 5 pills in the differing dosages appropriate for a patient, in different prescriptions and in different packages, the drugs are provided in one MUDP treatment regimen. Commonly prescribed dosages of S-D-A-T are employed in the MUDP treatment regimen formulations of the Primary Matrix.

In an embodiment, the first ingredient is either Aspirin 325 mg or PLAVIX 75 mg or both.

Aspirin is approved in one strength 325 mg and PLAVIX is approved in one strength 75 mg to keep blood platelets from sticking together. In an embodiment, the MUDP treatment regimen contains either 325 mg of aspirin or 75 mg of PLAVIX or both. PLAVIX (clopidogrel bisulfate) is an inhibitor of ADP-induced platelet aggregation acting by direct inhibition of adenosine diphosphate (ADP) binding to its receptor and of the subsequent ADP-mediated activation of the glycoprotein GPIIb/IIIa complex. Chemically it is methyl (+)-(S)-a-(2-chlorophenyl)-6,7-dihydrothieno [3,2-c]pyridine-5(4H)-acetate sulfate (1:1).

ACE Inhibitors block the formation of chemicals in the body that signal the body to increase blood pressure and increase heart rate in addition to the constriction of blood vessels. Beta blockers lower blood pressure.

To lower cholesterol, a patient can take a statin. Very few people need 80 mg of a statin. To lower blood pressure, a patient might take a diuretic, either 25 mg or 50 mg. Very few people would take 80 mg of a diuretic. A diuretic is the first line of defense to lower blood pressure. Diuretics help reduce excess water in blood and body tissue. High volumes of water cause the heart to pump harder, thereby increasing BP. An effective drug to lower blood pressure is an ACE

Inhibitor. Patients can take 10 mg, 20 mg, or 40 mg of an ACE Inhibitor. About one third of the population has elevated triglycerides. To lower them, most patients can take a mid-level strength dose of TRICOR 160 mgs or a high strength of TRICOR 200 mg and a few take the small dose 67 mg. TRICOR reduces triglycerides in the blood by altering blood sugar levels. The strength of each drug is dependent on which drug in the class of drugs is used.

In an embodiment, the MUDP treatment regimen has aspirin and/or clopidogrel. In an embodiment, the formulations of statins considered have strengths of 10-20-40 and perhaps 80 mg. The MUDP treatment regimen can have ZOCOR or LIPITOR but it is possible to use a treatment regimen with simvastatin or a range of other Ethical/Branded and Generic ACE Inhibitors can also be included in the treatment regimen. The ACE Inhibitor can be MONOPRIL, but others could be used are 10-20-40 mg also. In an embodiment, the diuretic is spironolactone at 25 or 50 mg. Hydrochlorothiazide can be used that comes in 25, 50 mg, or LASIX that comes in 20, 40, and 80 mg. Other branded and generic drugs, as well as diabetic drug treatment can be used in the MUDP treatment regimen. Other dosage levels than those mentioned here can also be used.

The term “commonly prescribed dosages” means those dosages that are customarily used by a wide percentage of the population designated to receive the particular drug. The term “unitized” means that the physician would recognize the typical unit dosage such as a statin at 10, 20, 40, 80 mg or any other dosage level that may be necessary for the patient. For example, in an embodiment, each MUDP treatment regimen formulation has a unitized dosage of the medicament in that the dosage listed “S10” is readily recognizable as a widely prescribed statin 10 mg dosage.

Spironolactone (common brand name ALDACTONE) and Triamterine are medications commonly known in medical practice as “potassium sparing” diuretics. Diuretics are used to remove a surplus of fluid from the body's bloodstream or tissues. It also acts as an aldosterone inhibitor (inhibits salt retention), and is used to treat advanced heart failure when symptoms persist after other drug therapies are maximized. When it is used in this manner, it is not used as a diuretic to remove extra fluid from the body.

Hydrochlorothiazide is a diuretic and antihypertensive. It is the 3,4-dihydro derivative of chlorothiazide. Its chemical name is 6-chloro-3,4-dihydro-2H-1,2,4-benzothiadiazine-7-sulfonamide 1,1-dioxide. Its empirical formula is C₇H₈ClN₃O₄S₂.

LASIX is a diuretic which is an anthranilic acid derivative. LASIX for oral administration contains furosemide as the active ingredient and the following inactive ingredients: lactose monohydrate NF, magnesium stearate NF, starch NF, talc USP, and colloidal silicon dioxide NF. Chemically, it is 4-chloro-N-furfuryl-5-sulfamoylanthranilic acid. Furosemide is available as white tablets for oral administration in dosage strengths of 20, 40 and 80 mg.

### Overview of the Diagnostic and Prescriptive Criteria

In an embodiment, upon completion of the diagnosis and evaluation of the patients Cardiovascular and Cerebrovascular risk factors, as either recommended and delineated by the clinical protocol described or independently established by the diagnosing authority, the patient's customized risk parameters are established. These risk parameters specifies the patient's risk category as delineated by the clinical protocol used. In an embodiment, an objective of the clinical protocol is to reduce the Risk category to the “Lowest Possible Risk” for a Cardiovascular or Cerebrovascular event as specified by the clinical protocol or any other quantitative or qualitative system of measurement of risk factors.

In an embodiment, the Risk Factors delineate the patient's requisite therapeutic regimen as prescribed by the clinical protocol in the form of a MUDP. In an embodiment, the combinatory drug compounds prescribed are in accordance with the guidelines established by the US National Institute of Health (NIH), The Framingham Heart Study, US Center for Disease Control and Prevention (CDC), The US Food and Drug Administration (FDA), the American Heart Association (AHA), The American Medical Association (AMA), the American Diabetic Association (ADA), The United Sates Pharmacopeia, any other governmental or recognized entity or institution in the United Sates or other countries of the world, and/or any prescriptive guidelines utilized by the physician for the implementation of the clinical protocol in whole or in part.

The MUDP as delineated by the appropriate section of the clinical protocol prescriptive matrices (or any additions or changes approved by the criteria established above) empowers the disease management authority (physician, or other entity) to customize the patient's dose for each component of the MUDP.

### Optional Genetic Testing Component of the Clinical Protocol Relating to the Variation of Human Genes as Applicable to Drugs Utilized by the Clinical Protocol

Note that any genetic methodology or diagnostic criteria currently available or that may become available toward establishing an elevated level of diagnostic and/or prescriptive criteria can be used with the subject invention. For example, in an embodiment, the clinical protocol therapeutic regimen is in accordance with the recommendations of the NIH relating to the prescribing of PLAVIX (clopidogrel) for cardiovascular and cerebrovascular patients.

In an embodiment, the clinical protocol recommends adherence to the prescriptive guidelines established by the NIH for PLAVIX as it relates to the Gene known as CYP2C19. In an embodiment, in the presence of a variation/mutation of the Gene CYP2C19, clinical protocol recommends use of Aspirin alternately or in combination with clopidogrel (PLAVIX), as advised by the prescribing authority.

TABLE 8
Statin Drug Table (examples)

| Trade Name | Generic Name | Sponsor |
|---|---|---|
| PRAVACHOL | Pravastatin | Bristol-Myers Squibb |
| MEVACOR | Lovastatin | Merck |
| ZOCOR | Simvastatin | Merck |
| LESCOL | Fluvastatin | Novartis |
| LIPITOR | Atorvastatin | Pfizer |
| BAYCOL | Cerivastatin | Bayer |
| CRESTOR | Rosuvastatin | Astra-Zeneca |
| ADVICOR | Lovastatin + extended | Kos Pharmaceutical |
| | Release Niacin | |

In an embodiment, elevated blood pressure, the most prevalent risk factor, is identified as a “primordial risk factor” and is targeted by the MUDP treatment regimen with the chemotherapeutic management tool of a Diuretic and an ACE inhibitor. This is represented in the matrices described from the most prevalent dosage used to the least prevalent dosage used. This is categorized by row in each of the 4 columns.

In an embodiment, elevated cholesterol, the next primordial risk factor, is targeted by the use of a statin and is listed in 3 groups of 6 rows based on the most prevalent dosage used to the least prevalent dosage used.

In an embodiment, elevated triglycerides, the third most often treated risk factor, is listed in order of prevalence from left to right in columns A, B and C. TRICOR (Fenofibrate) is not prescribed in all cases shown, thus the need for column D.

In an embodiment, to aid in making the matrix easy to use, color coding can be employed. For example, the least prescribed drug strengths, for statins, i.e. 80 mg, and diuretics, also 80 mg, can be listed in an orange segment, rows 19 to 36 (matrix range D19-D36-A36-A19). The bottom orange segment might appear out of order, but ranks use according to Blood Pressure and then by most frequent matching Statins.

In an embodiment, aspirin can be prescribed at the dose of 325 mg or any dose level for the patients needs. In an embodiment, to facilitate the physician in selecting one of the many MUDP treatment regimens, Matrix I uses color coding of groups or pluralities of selected ones of the MUDP treatment regimens. In an embodiment, the color code employed is: highest use is RED, 2nd highest use is BLUE, 3^{rd} highest use is GREEN, and least used MUDP Combination Medical Therapeutic Regimen is colored ORANGE.

TABLE 9
MUDP Treatment Regimen Matrix I
ASPIRIN or CLOPEDROGREL, plus, STATIN, DIURETIC,
ACE INHIBITOR, & With or Without TRICOR
Diabetic Treatment Drug(s) added for Patients with Type 2 Diabetes

| | A | B | C | D |
|---|---|---|---|---|
| 1 | S10-D25-A10-T160 | S10-D25-A10-T200 | S10-D25-A10-T67 | S10-D25-A10 |
| 2 | S10-D25-A20-T160 | S10-D25-A20-T200 | S10-D25-A20-T67 | S10-D25-A20 |
| 3 | S10-D25-A40-T160 | S10-D25-A40-T200 | S10-D25-A40-T67 | S10-D25-A40 |
| 4 | S10-D50-A10-T160 | S10-D50-A10-T200 | S10-D50-A10-T67 | S10-D50-A10 |
| 5 | S10-D50-A20-T160 | S10-D50-A20-T200 | S10-D50-A20-T67 | S10-D50-A20 |
| 6 | S10-D50-A40-T160 | S10-D50-A40-T200 | S10-D50-A40-T67 | S10-D50-A40 |
| 7 | S20-D25-A10-T160 | S20-D25-A10-T200 | S20-D25-A10-T67 | S20-D25-A10 |
| 8 | S20-D25-A20-T160 | S20-D25-A20-T200 | S20-D25-A20-T67 | S20-D25-A20 |
| 9 | S20-D25-A40-T160 | S20-D25-A40-T200 | S20-D25-A40-T67 | S20-D25-A40 |
| 10 | S20-D50-A10-T160 | S20-D50-A10-T200 | S20-D50-A10-T67 | S20-D50-A10 |
| 11 | S20-D50-A20-T160 | S20-D50-A20-T200 | S20-D50-A20-T67 | S20-D50-A20 |
| 12 | S20-D50-A40-T160 | S20-D50-A40-T200 | S20-D50-A40-T67 | S20-D50-A40 |
| 13 | S40-D25-A10-T160 | S40-D25-A10-T200 | S40-D25-A10-T67 | S40-D25-A10 |
| 14 | S40-D25-A20-T160 | S40-D25-A20-T200 | S40-D25-A20-T67 | S40-D25-A20 |
| 15 | S40-D25-A40-T160 | S40-D25-A40-T200 | S40-D25-A40-T67 | S40-D25-A40 |
| 16 | S40-D50-A10-T160 | S40-D50-A10-T200 | S40-D50-A10-T67 | S40-D50-A10 |
| 17 | S40-D50-A20-T160 | S40-D50-A20-T200 | S40-D50-A20-T67 | S40-D50-A20 |
| 18 | S40-D50-A40-T160 | S40-D50-A40-T200 | S40-D50-A40-T67 | S40-D50-A40 |
| 19 | S80-D25-A10-T160 | S80-D25-A10-T200 | S80-D25-A10-T67 | S80-D25-A10 |
| 20 | S80-D25-A20-T160 | S80-D25-A20-T200 | S80-D25-A20-T67 | S80-D25-A20 |
| 21 | S80-D25-A40-T160 | S80-D25-A40-T200 | S80-D25-A40-T67 | S80-D25-A40 |
| 22 | S80-D50-A10-T160 | S80-D50-A10-T200 | S80-D50-A10-T67 | S80-D50-A10 |
| 23 | S80-D50-A20-T160 | S80-D50-A20-T200 | S80-D50-A20-T67 | S80-D50-A20 |
| 24 | S80-D50-A40-T160 | S80-D50-A40-T200 | S80-D50-A40-T67 | S80-D50-A40 |
| 25 | S10-D80-A10-T160 | S10-D80-A10-T200 | S10-D80-A10-T67 | S10-D80-A10 |
| 26 | S10-D80-A20-T160 | S10-D80-A20-T200 | S10-D80-A20-T67 | S10-D80-A20 |
| 27 | S10-D80-A40-T160 | S10-D80-A40-T200 | S10-D80-A40-T67 | S10-D80-A40 |
| 28 | S20-D80-A10-T160 | S20-D80-A10-T200 | S20-D80-A10-T67 | S20-D80-A10 |
| 29 | S20-D80-A20-T160 | S20-D80-A20-T200 | S20-D80-A20-T67 | S20-D80-A20 |
| 30 | S20-D80-A40-T160 | S20-D80-A40-T200 | S20-D80-A40-T67 | S20-D80-A40 |
| 31 | S40-D80-A10-T160 | S40-D80-A10-T200 | S40-D80-A10-T67 | S40-D80-A10 |
| 32 | S40-D80-A20-T160 | S40-D80-A20-T200 | S40-D80-A20-T67 | S40-D80-A20 |
| 33 | S40-D80-A40-T160 | S40-D80-A40-T200 | S40-D80-A40-T67 | S40-D80-A40 |
| 34 | S80-D80-A10-T160 | S80-D80-A10-T200 | S80-D80-A10-T67 | S80-D80-A10 |
| 35 | S80-D80-A20-T160 | S80-D80-A20-T200 | S80-D80-A20-T67 | S80-D80-A20 |
| 36 | S80-D80-A40-T160 | S80-D80-A40-T200 | S80-D80-A40-T67 | S80-D80-A40 |

Color code boundary chart: high usage is red A1-B1-B6-A6; blue, second highest usage C12-C12-C12-A12; green 3rd highest usage D1-D18-D18-A-18; orange least used dosages D19-D36-A36-A19

### MUDP Treatment Regimen Matrix II

In an embodiment, elevated Blood Pressure, the most prevalent risk factor, identified by the MUDP treatment regimen protocol as a “primordial risk factor”, is targeted by the MUDP treatment regimen with the chemotherapeutic management tool of an ACE inhibitor without a diuretic. This option is for those physicians who choose not to use a diuretic for the management of elevated blood pressure, and is represented in the matrix from the most prevalent dosage used to the least prevalent dosage used. This is categorized by row in each of the 3 columns.

In an embodiment, elevated Cholesterol, the next primordial risk factor, is targeted by the use of a Statin and is listed in 4 groups of 3 rows based on the most prevalent dosage used to the least prevalent dosage used. The third most often treated risk factor, elevated Triglycerides, is listed in order of prevalence from left to right in columns E, F, and G. The least prescribed drug strengths, for statins, i.e. 80 mg, and diuretics, also 80 mg, are listed in the orange segment, rows 46 to 48. The bottom orange segment might appear out of order but ranks use by Blood Pressure and then by most frequent matching statins.

Color code for Matrix II: highest usage is RED, 2^{nd} highest usage is Blue, 3^{rd} highest use is Green, and least used is Orange.

TABLE 10
MUDP Treatment Regimen Matrix II
ASPIRIN or CLOPEDROGREL plus, STATIN,
ACE INHIBITOR, TRICOR
Diabetic Treatment Drug(s) added for Diabetic Patients

| | E | F | G |
|---|---|---|---|
| 37 | S10-A10-T160 | S10-A10-T200 | S10-A10-T67 |
| 38 | S10-A20-T160 | S10-A20-T200 | S10-A20-T67 |
| 39 | S10-A40-T160 | S10-A40-T200 | S10-A40-T67 |
| 40 | S20-A10-T160 | S20-A10-T200 | S20-A10-T67 |
| 41 | S20-A20-T160 | S20-A20-T200 | S20-A20-T67 |
| 42 | S20-A40-T160 | S20-A40-T200 | S20-A40-T67 |
| 43 | S40-A10-T160 | S40-A10-T200 | S40-A10-T67 |
| 44 | S40-A20-T160 | S40-A20-T200 | S40-A20-T67 |
| 45 | S40-A40-T160 | S40-A40-T200 | S40-A40-T67 |
| 46 | S80-A10-T160 | S80-A10-T200 | S80-A10-T67 |
| 47 | S80-A20-T160 | S80-A20-T200 | S80-A20-T67 |
| 48 | S80-A40-T160 | S80-A40-T200 | S80-A40-T67 |

Color code boundaries: red highest use E37-F37-F39-E39; blue 2nd highest use G37-G37-G42-E42; green 3rd highest use G43-G45-E45-E43; orange least used dosages E46-G46-G48-E48.

### MUDP Treatment Regimen Matrix III

In an embodiment, Matrix Three (3) is comprised of Aspirin or PLAVIX, plus various drug combinations to be considered to capture a wider share of the market at the fringe of the MUDP treatment regimen model. In an embodiment, Matrix Three combinations are for the occurrence of an individual risk factor or combinations of risk factors not commonly occurring.

TABLE 11
MUDP Treatment Regimen Matrix III
Elevated Blood Pressure Only
ASPIRIN or CLOPEDROGREL Plus: DIURETIC
and an ACE INHIBITOR
Diabetic Treatment Drug(s) added for Diabetic Patients

| | H | I | J |
|---|---|---|---|
| 49 | D25-A10 | D25-A20 | D25-A40 |
| 50 | D50-A10 | D50-A20 | D50-A40 |
| 51 | D80-A10 | D80-A20 | D80-A40 |

Elevated Blood Pressure and Elevated Triglycerides
ASPIRIN or CLOPEDROGREL Plus
DIURETIC, ACE INHIBITOR and TRICOR
Diabetic Treatment Drug(s) added for Diabetic Patients

| | K | L | M |
|---|---|---|---|
| 52 | D25-A10-T160 | D25-A10-T200 | D25-A10-T67 |
| 53 | D25-A20-T160 | D25-A20-T200 | D25-A20-T67 |
| 54 | D25-A40-T160 | D25-A40-T200 | D25-A40-T67 |
| 55 | D50-A10-T160 | D50-A10-T200 | D50-A10-T67 |
| 56 | D50-A20-T160 | D50-A20-T200 | D50-A20-T67 |
| 57 | D50-A40-T160 | D50-A40-T200 | D50-A40-T67 |
| 58 | D80-A10-T160 | D80-A10-T200 | D80-A10-T67 |
| 59 | D80-A20-T160 | D80-A20-T200 | D80-A20-T67 |
| 60 | D80-A40-T160 | D80-A40-T200 | D80-A40-T67 |

Elevated Blood Pressure and Elevated Triglycerides
ASPIRIN or CLOPEDROGREL Plus
ACE INHIBITOR and TRICOR
Diabetic Treatment Drug(s) added for Diabetic Patients

| | N | O | P |
|---|---|---|---|
| 61 | A10-T160 | A10-T200 | A10-T67 |
| 62 | A20-T160 | A20-T200 | A20-T67 |
| 63 | A40-T160 | A40-T200 | A40-T67 |

Elevated Blood Pressure and Elevated Cholesterol
ASPIRIN or CLOPEDROGREL Plus STATIN
and ACE INHIBITOR
Diabetic Treatment Drug(s) added for Diabetic Patients

| | Q | R | S |
|---|---|---|---|
| 64 | S10-A10 | S10-A20 | S10-A40 |
| 65 | S20-A10 | S20-A20 | S20-A40 |
| 66 | S40-A10 | S40-A20 | S40-A40 |
| 67 | S80-A10 | S80-A20 | S80-A40 |

Elevated Blood Pressure and Elevated Cholesterol
ASPIRIN or CLOPEDROGREL Plus
STATIN and DIURETIC
Diabetic Treatment Drug(s) added for Diabetic Patients

| | T | U | V |
|---|---|---|---|
| 68 | S10-D25 | S10-D50 | S10-D80 |
| 69 | S20-D25 | S20-D50 | S20-D80 |
| 70 | S40-D25 | S40-D50 | S40-D80 |
| 71 | S80-D25 | S80-D50 | S80-D80 |

Elevated Cholesterol and Elevated Triglycerides
ASPIRIN or CLOPEDROGREL Plus
STATIN and TRICOR
Diabetic Treatment Drug(s) added for Diabetic Patients

| | W | X | Y |
|---|---|---|---|
| 72 | S10-T160 | S10-T200 | S10-T67 |
| 73 | S20-T160 | S20-T200 | S20-T67 |
| 74 | S40-T160 | S40-T200 | S40-T67 |
| 75 | S80-T160 | S80-T200 | S80-T67 |

### MUDP Treatment Regimen Matrix IV

In an embodiment, Matrix Four (4) is comprised of Aspirin or Clopidogrel, plus various drug combinations to be considered to capture a wider share of the market at the fringe of the MUDP combination treatment model. In an embodiment, MUDP treatment regimen Matrix Four combinations are for the occurrence of combinations of risk factors not commonly occurring or treatment regimens not commonly prescribed in the United States.

TABLE 12
Elevated Blood Pressure, Cholesterol and Triglycerides
ASPIRIN or CLOPEDROGREL Plus:
STATIN, DIURETIC and an TRICOR
Diabetic Treatment Drug(s) added for Diabetic Patients

| | AA | BB | CC |
|---|---|---|---|
| 76 | S10-D25-T160 | S10-D25-T200 | S10-D25-T67 |
| 77 | S10-D50-T160 | S10-D50-T200 | S10-D50-T67 |
| 78 | S20-D25-T160 | S20-D25-T200 | S20-D25-T67 |
| 79 | S20-D50-T160 | S20-D50-T200 | S20-D50-T67 |
| 80 | S40-D25-T160 | S40-D25-T200 | S40-D25-T67 |
| 81 | S40-D50-T160 | S40-D50-T200 | S40-D50-T67 |
| 82 | S80-D25-T160 | S80-D25-T200 | S80-D25-T67 |
| 83 | S80-D50-T160 | S80-D50-T200 | S80-D50-T67 |
| 84 | S10-D80-T160 | S10-D80-T200 | S10-D80-T67 |
| 85 | S20-D80-T160 | S20-D80-T200 | S20-D80-T67 |
| 86 | S40-D80-T160 | S40-D80-T200 | S40-D80-T67 |
| 87 | S80-D80-T160 | S80-D80-T200 | S80-D80-T67 |

Elevated Blood Pressure and Elevated Triglycerides
ASPIRIN or CLOPEDROGREL Plus
DIURETIC, and TRICOR
Diabetic Treatment Drug(s) added for Diabetic Patients

| | DD | EE | FF |
|---|---|---|---|
| 88 | D25-T160 | D25-T200 | D25-T67 |
| 89 | D50-T160 | D50-T200 | D50-T67 |
| 90 | D25-T160 | D25-T200 | D25-T67 |
| 91 | D80-T160 | D80-T200 | D80-T67 |

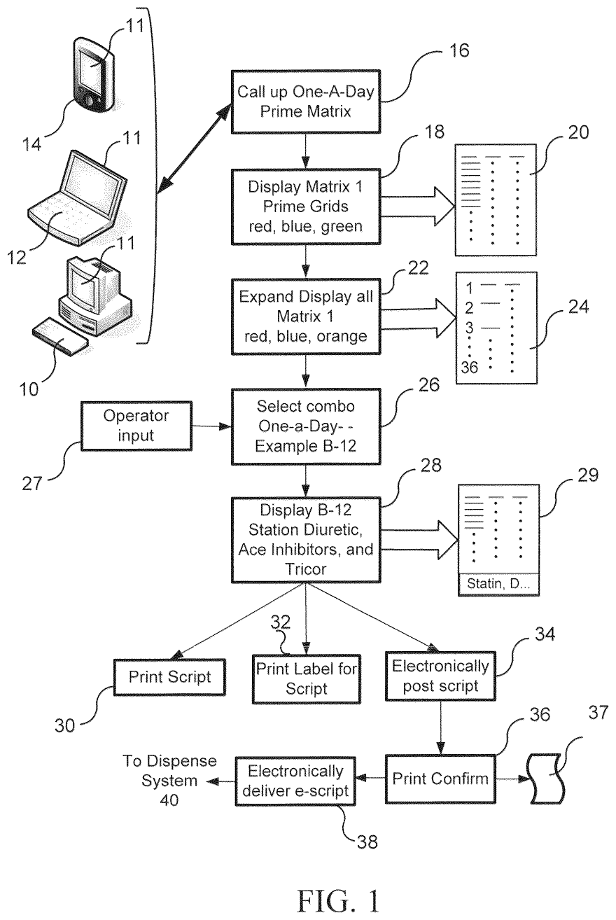

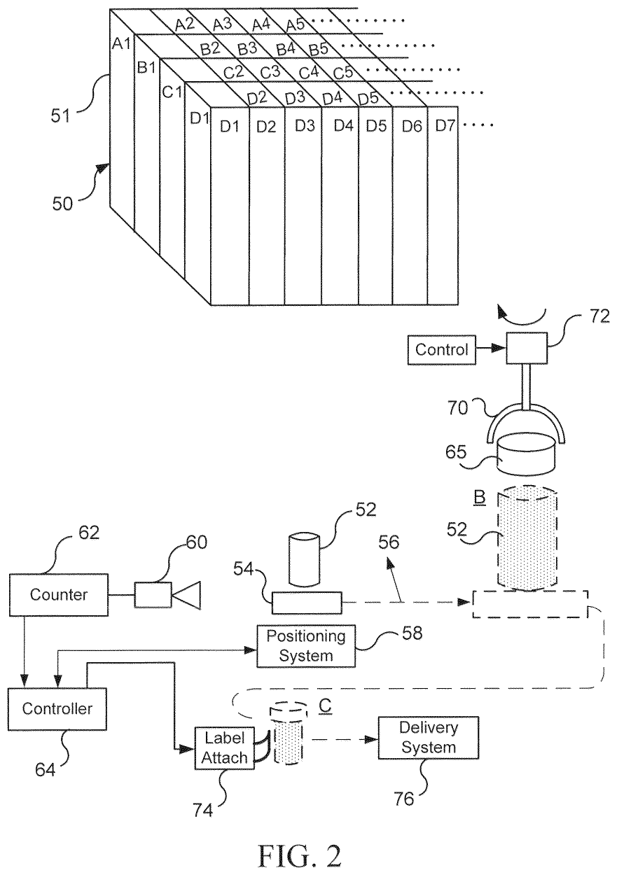

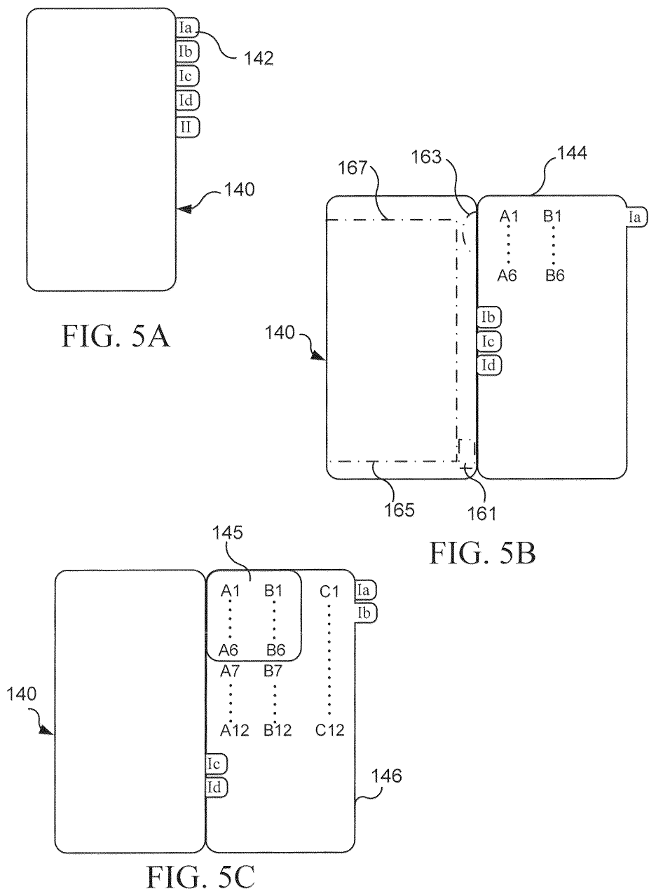

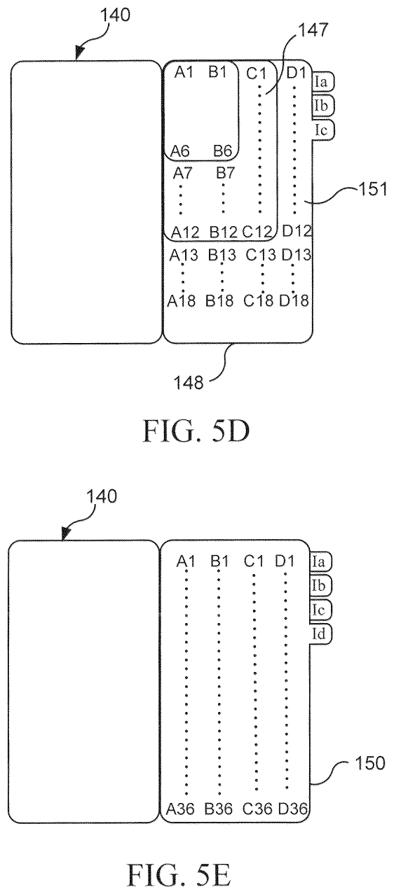

In an embodiment of the present invention, medicaments are delivered in a MUDP to be taken by the patient once a day or otherwise as prescribed. Previously, the patient would be prescribed 3-4 pills to be taken at various times during the day to improve the patient's health and reduce the patient's risk of CHD and CVD described above. In order to accomplish this selection activity by the healthcare provider, an information processing system is the subject of the present invention (shown in FIG. 1) as is a system for selection of one of the large plurality of MUDP treatment regimens (shown in FIGS. 5A-5E, 6A-6C and 7). Since the dispensing of a MUDP treatment regimen from a large plurality of MUDP treatment regimens is challenging, a dispensing system is diagrammatically illustrated in FIGS. 2-4. Similar numerals designate similar items throughout the drawings.

FIG. 1 diagrammatically illustrates an information processing system for selecting a treatment regimen from a plurality of treatment regimens in accordance with an embodiment of the subject invention. In an embodiment, each treatment regimen includes different formulations of commonly prescribed medicaments in commonly prescribed or unitized dosages. In an embodiment, at least some of the treatment regimens also include other prescribed activities such as tests or exercises. In and embodiment, the information processing system enables the physician to select one of a large plurality of MUDP treatment regimens. The information processing system can be employed over any type of computer system. In an embodiment, the computer system includes a memory, a display screen and operator input controls. As discussed above, various networks can be employed, including, but not limited to, Local area networks, wide area networks, wireless networks, and the Internet. In FIG. 1, a personal computer system 10 is shown, a laptop 12 is shown and a personal data assistant or PDA 14 is shown. These systems can have display screens 11 and include some type of operator input. The keyboard and mouse input on laptop 12 and on personal computer 10 may also include a touch screen input device as is common in PDA 14, display 11. In any event, in functional step 16, a physician, other healthcare provider, or other user refers to a data chart for the MUDP treatment regimen primary matrix. The MUDP treatment regimen Matrices I, II, III and IV can be stored in memory. Functional step 18 displays a matrix I (or a primary grid portion thereof). In an embodiment, the primary grids are displayed in red, blue, and green as described. The primary grids shown in red, blue and green colors are listed at treatment regimen Matrix I, column and row A-I, B-1, B-6 and A-6 (grid 1-red); secondary group in blue shown in matrix I at C-I, C-12, A-12 and A-7; and tertiary group without TRICOR in matrix I, matrix coordinates D-I, D-18, A-18 and A-13. This is shown diagrammatically in the figure as a matrix with subgroup red, blue and green 20. The display has matrix subgroups red, blue and green (see matrix boundary markers above) which enables the physician to select commonly prescribed medications, in commonly prescribed dosages, in a MUDP treatment regimen. Also, upon repeat visits, the physician can titrate dosages using the matrix to optimize the patient's health. Further, the healthcare provider can use the matrix with the patient as an educational and motivational tool to motivate the patient to titrate dosages to sub matrix or primary red grid AI, B1, B6, A6.

In functional step 22, the healthcare provider expands the display to include the entire MUDP Treatment Regimen Matrix I. In an embodiment, subgroups are displayed in red, blue, green, and orange. The display is shown in FIG. 1 as display 24 which includes the red grid, blue grid and green grid as well as the orange, least used group of treatment regimens from range A-19, D-19, D-36 and A-36. The information system can quickly shift displays from the primary grid to the secondary, tertiary and full matrix displays for titration purposes and educational purposes.

In functional step 26, the operator has an input (27) which selects one of the plurality of MUDP treatment regimens such as that listed at grid location B-12. As shown in the matrix I, above, at B-12, in the blue subgroup, the matrix display shows S-20-D50-A40-T200 which represents 20 mg of a statin, 50 mg of diuretic, 40 mg of ACE inhibitor and 200 mg of TRICOR/fenofibrate. The use of the abbreviations S-D-A-T also enables the physician to review many of the combinatory drugs and quickly select one. The indicia S-D-A-T greatly assists in the selection as does the mg dosage next to the letter indicia. In functional step 28, the system, upon operator selection, displays the full formula for the selected MUDP treatment regimen combinatory drug at grid location B-12. Display screen 29 is shown in FIG. 1. From functional step 28, the information processing system can provide various outputs, one of them being a print script or print prescription function 30, a second being a print label function showing the script as a label but not the entire script prescription pad at functional step 32, and also electronically posting the script at function 34. In function 36, the system prints a confirmation on paper 37 to show the selected one of the plurality of MUDP treatment regimens. In step 38, the system electronically delivers the electronic script to a dispensing system 40. Optionally, matrix grid portions maybe printed for reference or to motivate the patient.

With respect to the information processing system, the groupings of sub-pluralities of MUDP treatment regimen formulations can be stored in memory in one of the electronic devices 10, 12, and 14. Of course, a dumb terminal maybe utilized rather than a completely independent functional personal computer 10. Further, PDA 14 may be linked via wireless network connection to a main frame or server computer. The output generator is a combination of the touch screen display or the keypad or the mouse in electronic devices 10, 12, and 14 in addition to either a printer output or print script function 30 (also print label 32) or an electronic output as noted in electronic posting of the script in function 34. The output functions 30, 32, 34 may be local or near PDA 14 or maybe remote with respect to display screens 11. Or the function described can be otherwise distributed across a network as described above.

FIGS. 2-4 each diagrammatically illustrate a dispensing system for a plurality of medicaments in accordance with an embodiment of the subject invention.

With respect to the embodiment of FIG. 2, the dispensing system includes a plurality of containers 50 wherein each respective container, for example container 51, retains one MUDP formulation. For case explanation, these containers are marked A1, A2, A3, A4, A5, D1, D2, D3, D4, C1 . . . and B1 . . . . These labels are shorthand designations for the formulations in MUDP Matrix I and relate to the column and row designators. However, other types of designators maybe utilized including the S-D-A-T indicators. If the S-D-A-T indicia are used on the MUDP and used on storage containers 50, the dispensing operator could easily check and verify inventory. Since a large plurality of MUDPs is available to the consumer, a dispensing system confirming the selected MUDP is an objective of an embodiment of the present invention. Below each respective storage container 51 is a door or a MUDP dispensing opening with a control latch.

Supply container 52 is mounted, in the illustrated embodiment, on positioning trolley 54 which is adapted to move in the x-y direction 56 beneath the plurality of respective storage containers 50. A positioning system controller 58 controls positioner 54 and hence supply container 52. A sensing system 60 coupled to a counter detector 52 feeds control MUDP count signals into controller 64. Controller 64 also provides controlling signal to positioning system 58 thereby moving supply container 52 beneath the appropriate respective storage container 51. After the latch or door is open beneath the storage container 51 and MUDPs having a single formulation are dispensed into supply container 52 (subject to sensor 60 and counter 62), supply container 52 is moved to position B wherein cap 65 is attached to the supply container 52. Cap retainer 70 is rotated by drive motor 72 under a control signal from controller 64. Thereafter, supply container 52 with cap 65 is moved to position C wherein label attachment system 74 attaches the label that matches the respective storage container 51 established by controller 64. After position C, the supply container 52 is delivered to delivery system 76 which ultimately delivers the product to the consumer. Although a cylindrical container and cap are presented herein various shapes and configurations can be used for supply container 52. In an embodiment, a shape and configuration is chosen that is well adapted to hold the MUDPs used. In an embodiment, the supply container 52 is closed by means other than a cap which is twisted on.

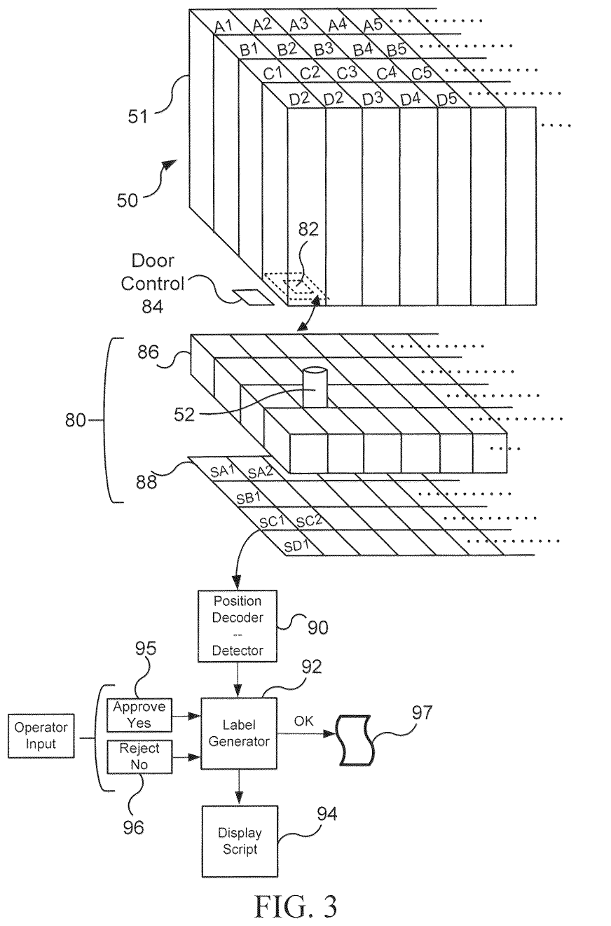

FIG. 3 diagrammatically shows another dispensing system having respective storage containers for each formulation (see plurality of storage containers 50), and a control dispensing interface 80 that differs from the dispensing interface in FIG. 2. The interface in FIG. 2 includes position system 58, positioner 54 and controller 64. It should be noted with respect to both FIG. 2 and FIG. 3, rather than moving the supply container 52, the plurality of storage containers 50 maybe moved. The important point is that the storage container for the selected MUDP is placed above or adjacent the supply container 52 such that the selected MUDP as ordered by the healthcare provider are dispensed from the respective container into the empty supply container 52. Intermediate hoses or chutes maybe interposed between supply container 52 and the chosen, selected storage container. The chutes or hoses maybe moved rather than the storage rack.

In FIG. 3, each of the respective storage chambers 50, each having a single MUDP formulation, includes a dispensing port 82 that is opened or closed via a door or latch under door control 84. Door control 84 is subject to control signals from a controller (not shown) similar to controller 64 in FIG. 2. In FIG. 3, the supply container 52 is put in an open top grid structure 86 which has beneath it a plurality of sensors 88. When the MUDPs from storage container C2 (for example) are loaded into supply container 52, sensor SC2 is activated thereby indicating that the MUDPs have been dispensed from dispenser storage container C2. Sensor SC2 may also weigh the supply container 52 to sense the condition (e.g., empty, partly full, or full) or number of MUDPs in the container. Position decoding detector 90 decodes the signal from the sensor grid 88. The output from positioned decoder/detector 90 is fed to label generator 92. The label generator is connected to a display unit 94 which shows the storage container which dispensed the MUDPs into supply container 52. The operator or pharmacist operating the dispensing system in FIG. 3 should confirm that the displayed formulation on screen 94 conforms to the written prescription related to the patient. The operator then provides an input to approve the MUDP dispensing or reject the dispensing operation as noted in operator input 95, 96. If rejected, a label is not generated and the MUDPs are not released. If the dispensing process has been approved by the pharmacist or healthcare worker, a label 97 is generated by label generator 92. The label is then attached to supply container 52. An automated label attachment sub-system maybe incorporated with label generator 92.

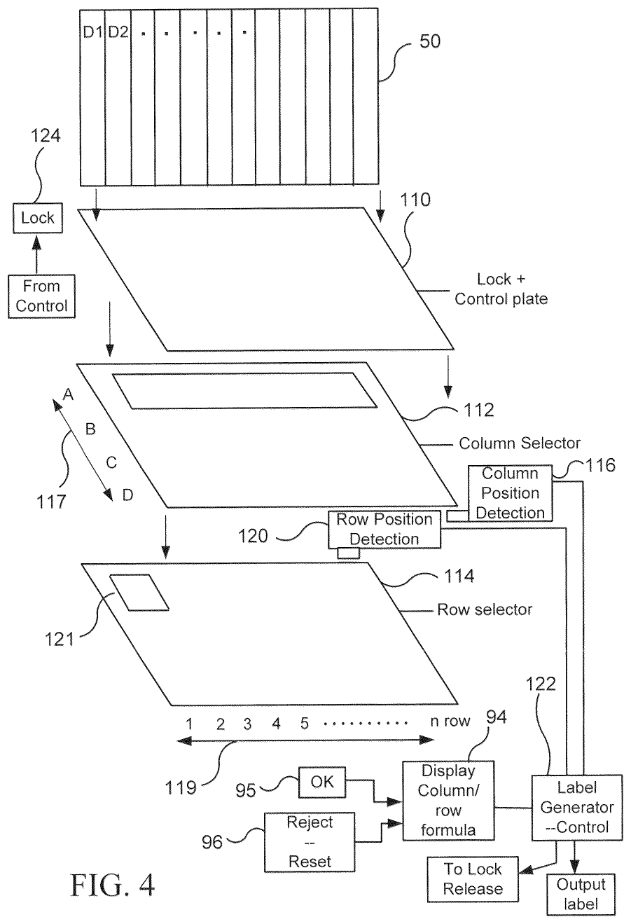

FIG. 4 shows another dispensing system which includes a three panel controlled MUDP dispensing interface consisting of lock and control plate 110, column selector plate 112 and row selector plate 114. In operation, a control unit (similar to control unit 64 in FIG. 2) moves column selector 112 to the appropriate column A, B, C, D (or otherwise) and the position of column selector 112 is detected and recorded by column position detector 116. The opening in column plate 112 is placed beneath the selected storage container. The column selector moves in the direction of double headed arrow 117. Row selector 114 is then moved to the particular row such as row and column D2 and under the selected storage container from the plurality of containers 50. Row selector 114 moves in direction shown in double headed arrow 119. The position of row selector 114 is detected by row position detector 120. When the correct column and row is detected by position detectors 116, 120, these data signals are applied to label generator and control 122. The label generator and control 122 generates a lock release signal which is applied to lock unit 124. Lock unit 124 then releases the lock and control plate 110 and moves the lock and control plate away from the plurality of MUDP storage containers 50. Otherwise, lock plate may be moved manually away to expose the open row and column. This permits the one selected storage container to dispense MUDPs since a particular row has been selected by row selector 116 and column has been selected by column selector 112. Label generator and control 122 generates a label for the supply container which is located beneath space 121 in row selector 114. As a safety precaution, display unit 94 lists the row and column as well as the selected formula. The healthcare provider must approve or reject the selected row and formula prior to the release of lock 124, the movement of control plate 110 and the production of the label from the label generator control 122.

FIGS. 5A-5E and 6A-6C and 7 show various systems for selecting one of the plurality of MUDP treatment regimens from various substrates which display various matrices and portions of Matrix I. See the red, green, blue and orange subgroups in Matrix I discussed above. In FIG. 5A, booklet 140 has a plurality of tabs 142 which extend from the side, top or bottom. Each tab is labeled with an identifier for the subgroup of treatment regimen Matrices such as Matrix la which distinguishes the subgroup at MUDP treatment regimen Matrix I grid coordinates A-I, B-1, B-6and A-6. Tab Ib shows the matrix of the first and the second matrix subgroups (red and green) thereby showing the physician grid group AI, C1, C12 and A12. By providing a selection system which lists the treatment regimens in a particular subgroup, this greatly assists the physician or healthcare provider to select one of the identified treatment regimens and also provides a motivational tool for the patient to move from one lower grid into a higher quality grid such as moving from matrix grid region Ib to grid region Ia. Color coding also provides patient motivation, patient education and dosage titration assistance to the physician. Since titration or the gradual change of medication dosage is contemplated by this invention, grid subgroups and matrix displays are quite helpful to the physician. FIG. 5B shows booklet 140 with a pullout sleeve 144 associated with the tab la. The printed subpart substrate shows the primary grid AI, B1, B6 and A6 MUDP treatment regimen formulations. FIG. 5C shows a second printed subpart substrate with the treatment regimen grid pattern C1, C12, A12 and A7 (secondary sub-matrix). A window 145 on printed substrate 146 shows the underlying or primary grid AI, B1, B6, A6 which is the lowest formulation for the MUDP treatment regimen.

FIG. 5D shows that subpart substrate 148 includes a larger cutout 147 which shows the second grid matrix Cl, C12, A12, A7. The next larger grid matrix D1, D18, A18, A13 (tertiary submatrix) is printed as indicia on surface 151 of subpart substrate 148. Windows or openings in substrates 144, 146 permit the user-healthcare provider to titrate dosages.

In FIG. 5E, the printed substrate 150 shows the entire matrix I. This permits the doctor to titrate dosages and show these dosages as he titrates the medication through the various grid levels.

The substrates shown in FIGS. 5A-5E are movable with respect to each other based upon outboard tabs 161, 163, slidably movable in slots 165, 167. The tab in slot slide system in FIGS. 5A-5E operates on substrates 144, 146. Other movable systems permitting movement of the printed substrates having indicia of the subpluralities and formulations can be utilized.

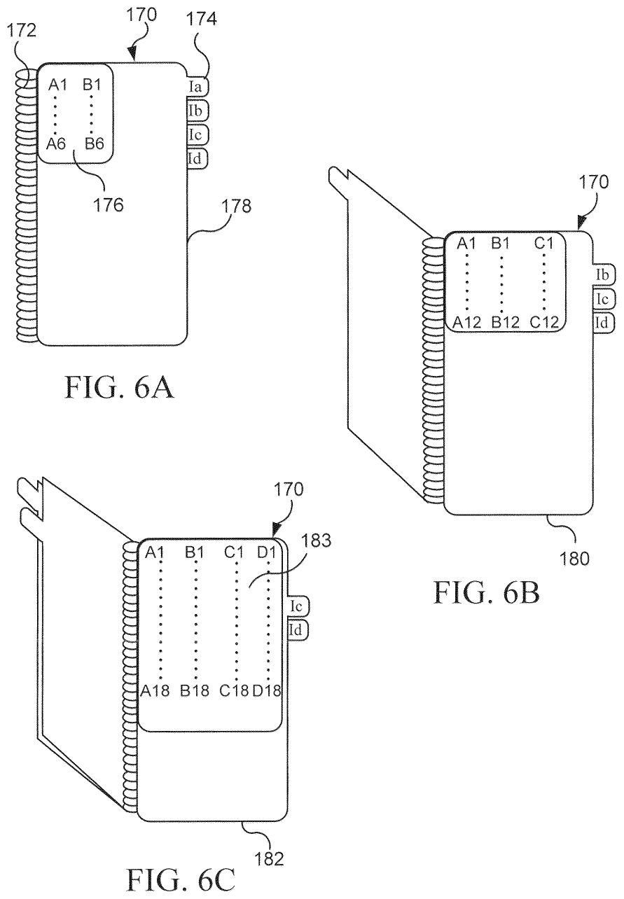

FIGS. 6A-6C show a booklet 170 with hinges, spiral, or other binding 172. The bound substrates have tabs 174 each marked with indicia representing both the MUDP treatment regimen matrix as well as the subpart of the matrix. Therefore, matrix sub grid Ia is displayed by the use of tab 174 and primary sub grid pattern AI, B1, B6, A6 is shown in window 176 of substrate card 178. With respect to FIG. 6B, substrate card 180 is keyed to secondary sub matrix grid portion 1b which shows grid pattern AI, C1, C12, A12. Card 182 associated with the indicia for tertiary matrix sub-group Ic has a cutout or window 183 permitting the view of sub grid AI, D1, D18, A18. A similar system can be employed for the other matrixes and sub grids.

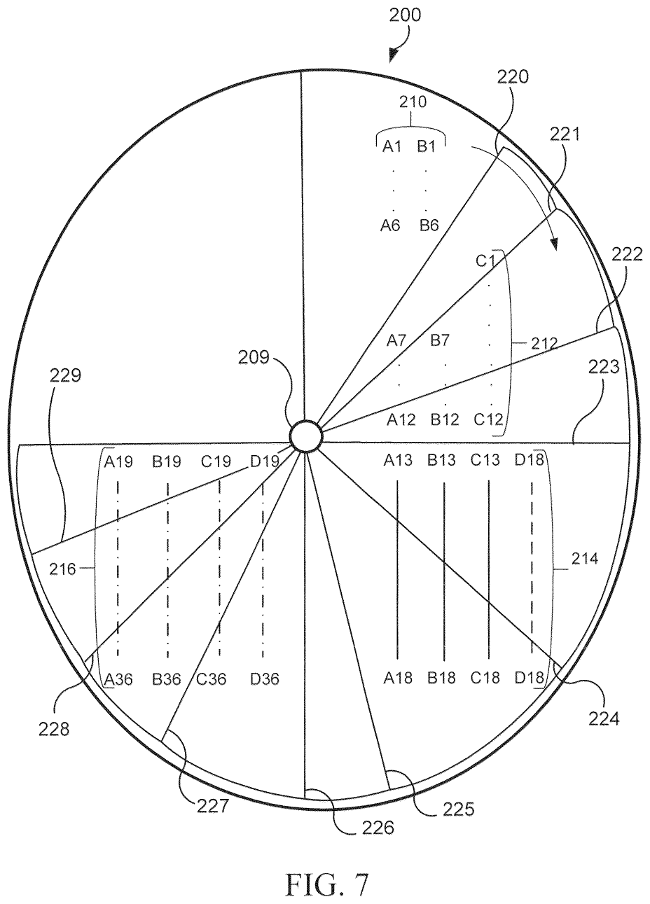

The selection system shown in FIG. 7 is a generally circular or oval substrate 200 having printed thereon portions of Matrix I. In region 210, indicia representing the first sub grid AI, B1, B6, A6 is shown. In region 212, the sub grid C1, C12, A12, A7 is shown. In substrate region 214, sub grid A13, D1, D18, A18 is shown. In substrate region 216, treatment regimens at subgrid A19, D19, D36, A36 are shown. Movable slides 220-229 cover all or substantially all of these grid indicia. In order to expose substrate portion 210, as well as substrate portion 212, rotatable fan collapsible elements 220, 221, 222, and 223 collapse on top of each other about rotation point 209. In order to expose the sub grid region and printed sub grid portion 214, slidable elements 220-226 are slid and rotated such that they lay adjacent on top of each other. In order to expose the entire grid, the user moves slidable elements 226, 227, 228, and 229 thereby exposing all of Matrix 1.

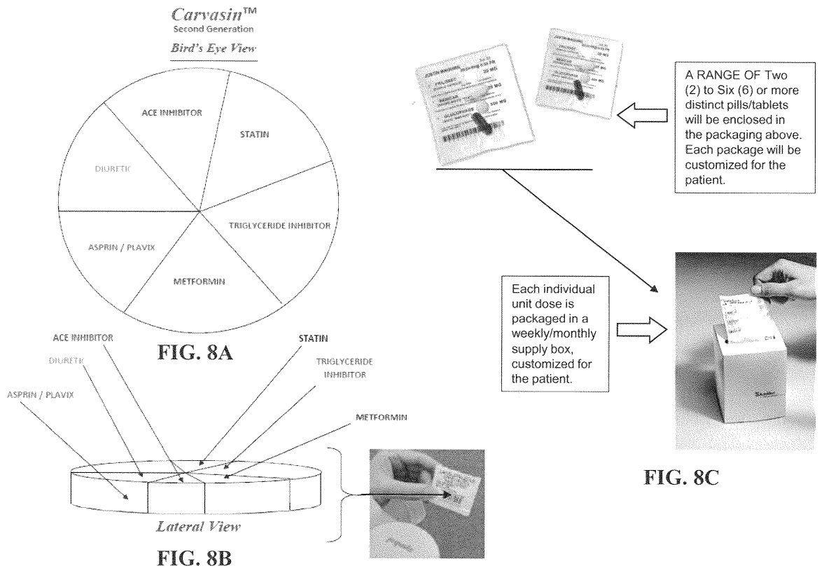

FIGS. 8A-8C illustrate various apparatuses for packaging multiple medicaments for convenient dosing according to embodiments of the subject invention. FIG. 8A illustrates a bird's eye view (top view) and FIG. 8B illustrates a lateral view of a packaging apparatus for a medicament according to an embodiment of the subject invention. The representation illustrates the separation of each pharmaceutical into a “pie-shaped” section or pocket. In an embodiment, each pharmaceutical pill or tablet is separated from the adjacent pill or tablet by a thin plastic partition and the whole packaging clement comprises a Multi Unit Dose Package, or “MUDP.” In FIG. 8C, the MUDPs comprising the customized and separated pharmaceutical prescriptions are packaged individually and then packaged into a multi-unit supply box for delivery to the patient. In the embodiment shown, the MUDPs can include two to six pills and the supply box includes a week or month-long supply. In another embodiment, the MUDPs can include one pill comprising multiple medicaments. In another embodiment, the MUDPs can include more than six pills. In another embodiment, the supply box can include less than a week's supply. In another embodiment, the supply box can include more than a week's supply. In a particular embodiment, the supply box contains a three-month supply of medicaments.

In an embodiment, the subject invention comprises a packaging system intended to increase patient compliance and improve efficacy of treatment for disorders such as hypertension, hypercholesterolemia, hypertriglyceridemia, anti-platelet aggregation, type 2 diabetes and related health issues, among other medical conditions or health problems. As shown in FIG. 8, the packaging system includes drug treatments such as aspirin and/or clopidogrel (also known as PLAVIX), diuretics, ACE inhibitors, statins, triglyceride inhibitors, and metformin and/or DIABENESE (Chlorpropamide), and/or any other drug compound that may manage the Diabetic Risk Factor(s) created by hyperglycemia and/or Insulin resistance. The packaging system enables the physician or healthcare provider to deliver a plurality of pills in a “Multi Unit Dose Package” (MUDP). The packaging system of the subject invention comprises a pouch that in embodiments has multiple indented “pie-shaped” pockets. Each pic-shaped pocket is separated by the plastic packaging material of the MUDP. The packaging system can contain multiple pills, preferably in different colors, in the pic-shaped arrangement that can appear as one multicolored pill when packaged. Therefore, the physician or healthcare provider can prescribe multiple pills based on the determined customary therapeutic needs of the individual under treatment based on a clinical protocol and/or combination therapy matrices. The MUDP treatment regimens thus deliver a complete drug treatment dosage in one easy-to-use package that greatly increases patient drug therapy compliance. Concurrent use of the clinical protocol and combination therapy matrices described also permit simplified titration of dosages to create new drug treatment regimens as a patient's needs change. To accommodate the changing therapeutic needs, new or titrated MUDP treatment regimens may be readily created for delivery to patients without disruption to the patient's level of compliance in the drug therapy.

FIG. 8 also illustrates methods of delivery for the MUDP treatment regimen supply boxes over weekly, monthly, quarterly, or longer periods of time based upon the condition of the patient. The multi-day treatment supply boxes comprising MUDP treatment regimens based on a prescribed drug treatment regimen are thus packaged to further improve patient compliance. The use of a MUDP treatment regimen for the individual's pills with various commonly prescribed dosages of preventive medications greatly enhances compliance. Most people remember to take a single dose each day. Similarly, the combination of pills into a MUDP treatment regimen can greatly increase compliance and the overall health of the patient.

Embodiments of the present invention relate to a diagnostic criteria, medical regimen prescribing system, and packaging systems aimed at improving patient compliance with prescribed drug treatment regimens. Embodiments of the subject invention are meant to target various disorders, conditions, health issues, and/or physiological systems including, but not limited to: Cerebrovascular and Cardiovascular Systems; Diabetes; Gastrointestinal System (GI); Urogenital System, including the Gaul Bladder, and Kidneys; Respiratory System; Neurological System; Endocrine System; Reproductive System; Dermatologic Diseases; and/or Electrolyte and Fluid Systems of the Body. In embodiments, the packing, distribution, and/or compliance monitoring systems of the subject invention can be used to package, distribute, and/or monitor compliance with various commonly used medicaments. In a particular embodiment, the invention is used to package, distribute, and/or monitor compliance with birth control medicaments.

Either the multi-pocketed pouch or pouch containing multi drug combinations of the subject invention creates the “Multi Unit Dose Package.” The Multi Unit Dose Package permits the easy prescription of various drugs at interchangeable dosages by the physician or health care provider. The Multi Unit Dose Package (MUDP) also improves patient compliance (an issue with many patients in multi-drug treatment) with the prescribed drug treatment regimen. The packaging system and MUDP also permits the ready packaging and delivery of long-term drug treatment by using MUDP supply boxes. The long-term packaging also works to improve patient compliance with drug treatment.

The following diagnostic criteria is an example of a diagnostic criteria which can be used with the subject invention; however, the systems and methods of the subject invention can be implemented with other diagnostic criteria. In an embodiment, the objective of the diagnostic criteria is to reduce and maintain the patient's Cardiovascular and Cerebrovascular Risk to a “low risk state.” Various definitions can be used for the low risk state. In an embodiment, the low risk state is defined as indicated in table 13.

TABLE 13
DEFINITION OF A LOW RISK STATE

| | |
|---|---|
| Total Serum Cholesterol | 160-199 mg/dL |
| Low Density Lipoproteins - C | 100-129 mg/dL |
| High Density Lipoproteins - C | 45 mg/dL in Men and 55 mg/dL. In |
| | Women |
| Blood Pressure | ≤115 mm Hg. Systolic and ≤75 mm |
| | Hg. Diastolic |
| Nonsmoker | Maintain abstinence |
| Control of Diabetes | HbA1c ≤6% and Glucose levels |
| | between 70 mg and 150 mg. |
| Blood Triglycerides | ≤150 mg/dL |

Other definitions of the low risk state can be selected by one skilled in the art and used with the subject invention.

Various diagnostic modalities can be used with the subject invention to assess patient health. By way of example, the following modalities, among others, can be used:
1. Patient's Age;
2. ECG and EKG abnormalities;
3. Noninvasive Tests of Atherosclerotic Burden;
4. Ankle-Brachial Blood Pressure Index;
5. Arterial B-Mode Ultrasound;
6. Status of Aortic atherosclerosis;
7. Status of Total Serum Cholesterol;
8. Status of Low Density Lipoproteins-C;
9. Status of High Density Lipoproteins-C;
10. Blood Pressure;
11. Control of Diabetes;
12. Blood Triglycerides;
13. Status of Homocysteine;
14. Imaging of the Heart, Brain, Aorta, Carotids, and other organs including, but not limited to:
   a. CT Scan;
   b. MRI; and
   c. Ultrasound.
      In an embodiment, CHD is managed in accordance with severity of condition determined by imaging including, but not limited to, Plaque formation, constriction of artery, etc.

In an embodiment, various risk factors are assessed as part the diagnostic criteria. Such risk factors include, but are not limited to, predisposing risk factors, conditional risk factors, and controllable risk factors. Nonexclusive examples of these types of risk factors are provided in table 14 and 15.

TABLE 14

| PREDISPOSING RISK FACTORS | CONDITIONAL RISK FACTORS |
|---|---|
| Obesity | Elevated Serum Triglycerides |
| Abnormal Obesity | Elevated LDL particles |
| Physical Inactivity | Elevated Serum Homocysteine |
| Family History of Premature | Elevated Serum Lypoprotein “a” |
| Heart Disease | |
| Ethnic Characteristics | Prothrombotic Factors (i.e. |
| | Fibrinogen) |
| Psychosocial Factors | Inflammatory Markers (i.e. |
| | C-Reactive Protein) |

TABLE 15
CONTROLLABLE RISK FACTORS

| VITAL FACTOR | MANAGEMENT |
|---|---|
| High Blood Pressure | ≤115 mm Hg. Systolic and ≤75 mm Hg. |
| | Diastolic |
| Abnormal Cholesterol | 160-199 mg/dL. |
| Abnormal Triglycerides | ≤150 mg/dL |
| Tobacco Use | Abstinence of tobacco use: |
| | Health Insurance Incentives |
| | Family and Community Involvement |
| | Governmental Support |
| Diabetes | HbA1c <6% |
| | Glucose levels between 70 mg and 150 mg |
| Overweight | Body weights are currently defined |
| | according to BMI as follows: |
| | Normal weight 18.5-24.9 Kg/m² |
| Physical Inactivity | Daily exercise |

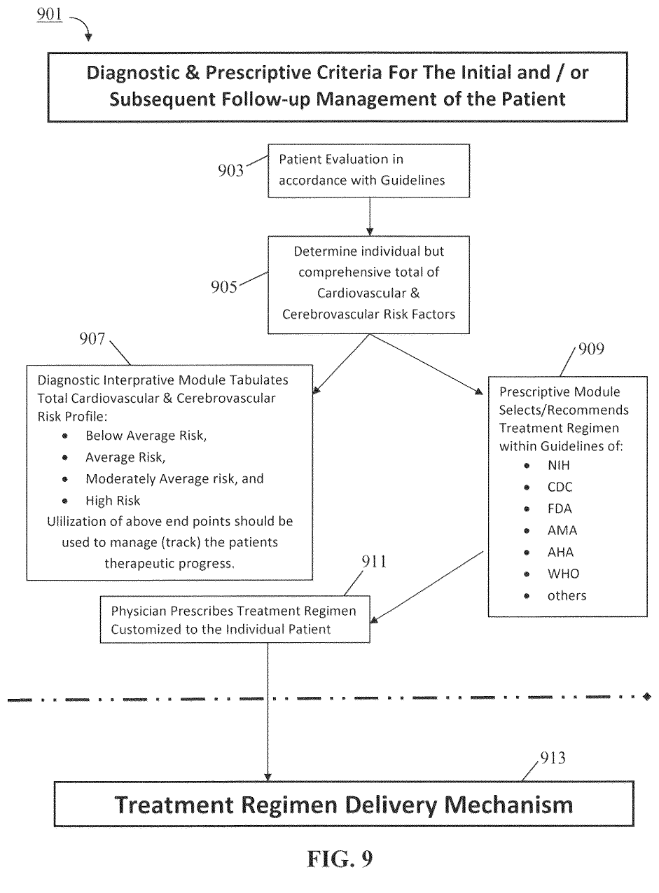

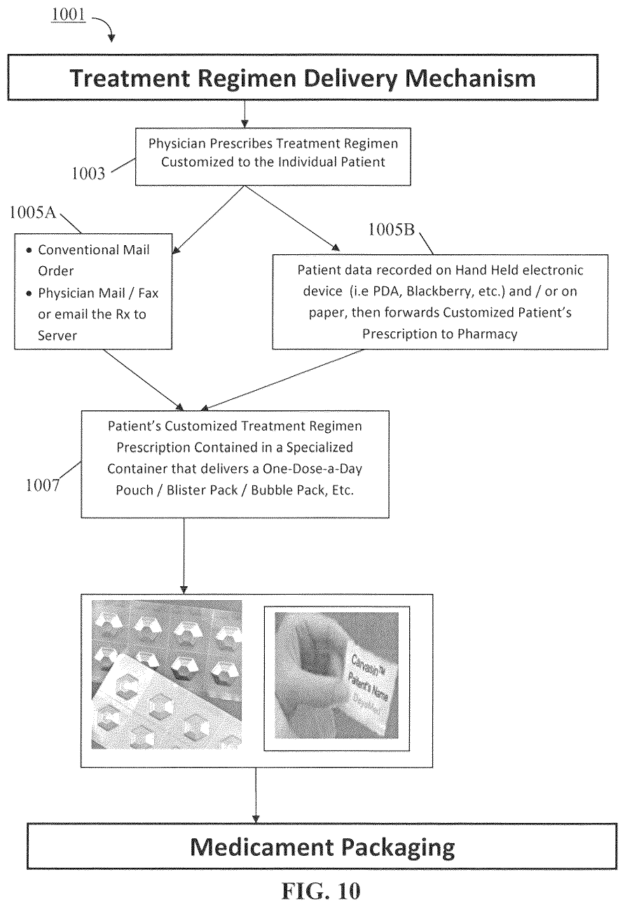

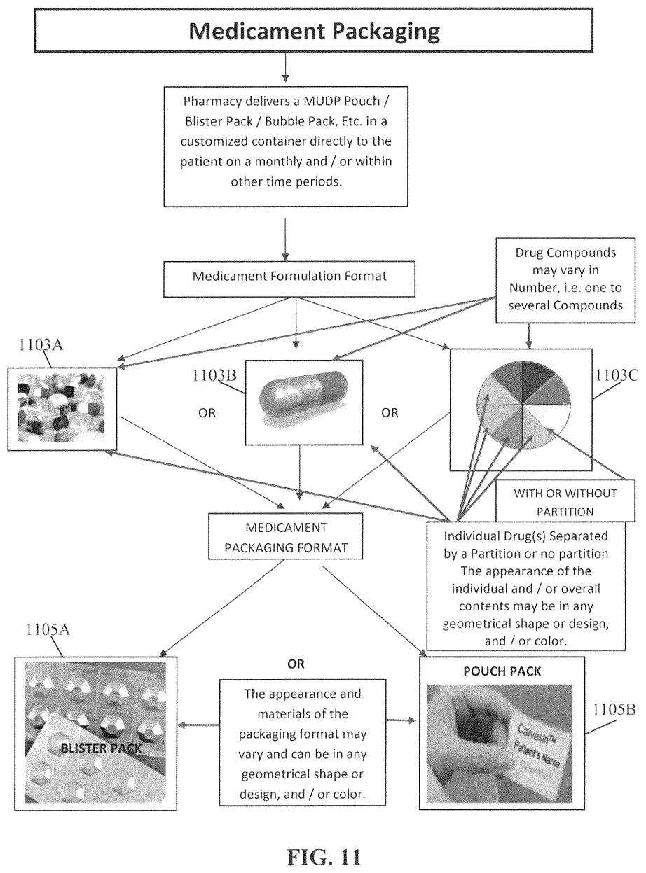

FIGS. 9-11 illustrate methods and apparatuses for disease management according to embodiments of the subject invention. FIG. 9 illustrates a method for diagnosing a disease in a patient and selecting a treatment regimen in accordance with an embodiment of the subject invention. FIG. 10 illustrates a method for delivering a medicament to a patient in accordance with an embodiment of the subject invention. FIG. 11 illustrates example apparatuses for packaging one or more medicaments for delivery to a patient in accordance with an embodiment of the subject invention. These methods can be used separately or linked together as indicated.

In an embodiment of the subject invention, the method 901 of FIG. 9 is used to diagnose and select a treatment regimen for a patient. As indicated the method 901 can be used for initial diagnosis of disease or subsequent follow-up. In an embodiment, the method 901 is used in primary care of the patient. In another embodiment, the method 901 is used in secondary care of the patient. At a step 903, the patient is evaluated in accordance with various guidelines. As further discussed below, a Diagnostic Module, such as Diagnostic Module 1203, can be used to collect and evaluate various information about the patient (“patient information”). At a step 905, the patient's risk factors for disease are determined. Again, although cardiovascular and cerebrovascular risk factors are indicated here, the method 901 and other embodiment of the invention can be used to diagnose and treat other diseases or conditions. Regardless, at a step 907, a Diagnostic Interpretive Module, such as Diagnostic Interpretive Module 1205 described below, can be used to evaluate risk factors and thereby assess the patient's disease risk. As indicated, this step can be used to determine the patient's initial disease risk as well as to track the patient's progress in reducing such risk. At a step 909, a Prescriptive Module, such as the Prescriptive Module 1207 described below, is used to select and/or recommend a treatment regimen based on some or all of the patient information and guidelines. At a step 911, a physician or other healthcare provider prescribes a treatment regimen based on the selection/recommendation or the step 909. In an embodiment, a plurality of treatment regimens are recommended in the step 909 and physician selects one of the recommended treatment regimens in the step 911. In another embodiment, a single treatment regimen is selected in the step 909 and the physician confirms, modifies, titrates, or otherwise customizes the selected treatment regimen in the step 911. In another embodiment, the physician is not consulted and the method 901 proceeds directly from the step 909 to the step 913. At a step 913, the selected treatment regimen is communicated to a treatment regimen delivery mechanism, such as the Dispensing Module 1209 described below, for delivery and/or communication to the patient. In an embodiment, the method 1001, described next, is part of a treatment regimen delivery mechanism.

In an embodiment of the subject invention, a method 1001 of FIG. 10 is used to deliver components of a treatment regimen to a patient. In an embodiment, the method 1001 is preformed by a Dispensing Module, such as the Dispensing Module 1209 described below. According to the method 1001, at a step 1003 a physician or other health care provider selects a treatment regimen for the patient. In an embodiment, the method 901 is used for this selection. In an embodiment, the selection is communicated at this point to the Dispensing Module. Next, the selected treatment regimen is communicated to a pharmacy, Dispensing Module, or other source of treatment components. As indicated by steps 1005A and 1005B, this communication can occur via various communication technologies known in the art. In a step 1007, the components of the treatment regimen are delivered to the patient or other location. In an embodiment, a dispensing apparatus, such as the dispensing apparatus 1301 described below, is delivered as part of the step 1007. In an embodiment, one or more of the example apparatuses illustrated in FIG. 11 are used to package medicaments included in the delivered treatment regimen.

FIG. 11 illustrates example apparatuses for packaging one or more medicaments for delivery to a patient in accordance with an embodiment of the subject invention. As shown, the medicament formulations can have various formats (e.g., 1103A-C) and the medicaments can be packaged in using various packing formats (e.g., 1105A-B). The various formats shown in FIGS. 8A-C can also be used with the subject invention. These formats are merely examples. Other formats can be used with the subject invention.

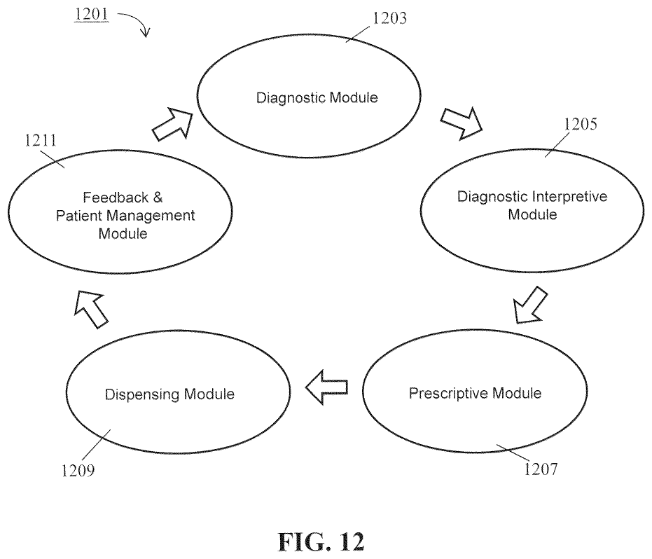

FIG. 12 diagrammatically illustrates a Disease Management System 1201 in accordance with an embodiment of the subject invention. In the embodiment shown, the Disease Management System 1201 includes a Diagnostic Module 1203, a Diagnostic Interpretive Module 1205, a Prescriptive Module 1207, a Dispensing Module 1209, and a Feedback and Patient Management Module 1211. Many different arrangements of the various components depicted, as well as components not shown, are possible without departing from the spirit and scope of the present invention. In embodiments, subsets of the components depicted are utilized. In embodiments, each component is used alone without the others depicted.

The arrows shown in the diagram of FIG. 12 illustrate a possible information flow between the components depicted. In other embodiments of the subject invention, information can flow in cither direction between any pair of components forming part of the Disease Management System 1201. In embodiments, intermediate components not shown can be included. In the embodiment shown, the information flows in a circle to complete a feedback loop as further described below.

In an embodiment of the subject invention, the Diagnostic Module 1203 provides access to patient information, such as the patient's chart, medical records, imaging, etc., as well as scientific guidelines for patient treatment. In an embodiment, the Diagnostic Module 1203 provides an interface that can be used to both read and write this type of information. In another embodiment, the Diagnostic Module 1203 only provides read access and the information is recorded via a different interface.

Various patient information can be viewed and/or recorded via the Diagnostic Module 1203. For example, among other patient information, the Diagnostic Module 1203 can provide access to:
1. Patient History, such as:
   a. Current Symptoms/Pathologies;
   b. Current Medications;
   c. Patient Identification Data;
   d. Race/National Origin;
   e. Education;
   f. Occupation;
   g. Family History/Composition;
   h. Life Style/Diet;
   i. Salt Intake;
   j. Fruits & Vegetables;
   k. Low Fat Dairy Products;
   l. Alcohol Intake;
   m. Physical Activity;
   n. Tobacco Use; and
   o. Other relevant historic information about the patient;
2. Physical Examination, such as:
   a. Height;
   b. Weight;
   c. Temperature;
   d. Blood Pressure;
   e. Heart rate;
   f. Respiration rate;
   g. Review of systems; and
   h. Other relevant exam results.
3. Blood Profile and Laboratory Tests, such as:
   a. CBC with Differential;
   b. Blood Glucose;
   c. 3 month Average Glucose;
   d. Hemotocrit;
   e. Lipid Panel;
   f. Serum Potassium;
   g. Creatinine;
   h. Calcium;
   i. Urine Analysis: Albumin/Creatinine Ratio;
   j. Homocysteine Profile; and
   k. Other relevant Laboratory Testing;
4. Imaging and Electronic Tests, such as:
   a. Ankle/Brachial BP;
   b. EKG;
   c. BMI;
   d. CT Scan;
   e. MRI;
   f. B-Mode Ultrasound (Intima/Media Thickness):
      i. Carotid;
      ii. Aorta;
      iii. Femoral Artery; and
   g. Other relevant imaging and electronic testing.

In an embodiment of the subject invention, the Diagnostic Interpretive Module 1205 provides tools to evaluate risk of particular diseases based on patient information and an evaluative methodology. In a further embodiment, the level of risk is categorized into predetermined categories. In an embodiment, the categories Below Average Risk, Average Risk, Moderately Above Average Risk, and High Risk are used. Other methods of categorizing disease risk will be known to those skilled in that art and can be used with the subject invention. Various evaluative methodologies known in the art can also be employed. In an embodiment, the risk factors and/or co-morbidities assessed include, but are not limited to, the presence of diabetes mellitus, hypertension, dyslipidemia, obesity, hypertriglyceridemia, tobacco use, microalbuminuria, target organ damage, sleep apnea, kidney disease, primary Aldosteronism, renovascular disease, Cushing's Syndrome, Pheochromocytoma, Coarctation of Aorta, as well as the patient's thyroid/parathyroid, Glomerular filtration rate, age, physical activity, and family history of CVD or premature CVD. In an embodiment, a subset of these risk factors and/or co-morbidities are assessed. In an embodiment, male patients over 55 and female patients over 65 are deemed to be at increased risk. Body Mass Index among other methods can be used to determine the presence of obesity. In an embodiment, the risk associated with hypertension is assessed according to the methodology described in Table 3 above. Other methodologies for assessing hypertension are known in the art and can be used with the subject invention.

In an embodiment of the subject invention, the Prescriptive Module 1207 is used to recommend, select, and/or evaluate one or more treatment regimens based on patient information and guidelines. In a further embodiment, the Prescriptive Module 1207 also transmits an indication of a selected treatment regimen to one or more persons involved in the care or treatment of the patient. For example, the Prescriptive Module 1207 can transmit an indication to the patient, one or more health care providers treating the patient, and/or one or more payors financing the treatment of the patient, among other persons or entities participating in the provision of the patient's care or health management. Payors can include persons paying directly for some or all of the care or treatment of the patient or those providing insurance or other coverage which pays for some or all of the treatment of the patient. In an embodiment, state or federal governments may be considered payors. The treatment regimen may include one or more medicaments, tests, activities, or other treatment to be taken by the patient. The indication of the treatment may describe some or all of the treatment regimen at various levels of detail depending on need. In a further embodiment, the Prescriptive Module 1207 also directs medicaments or other items needed for the treatment regimen to be delivered to the patient. In an embodiment, the Prescriptive Module 1207 directs a dispensing device, such as the Dispensing Device 1303 described below, to be delivered to the patient.

In an embodiment, the Prescriptive Module 1207 recommends one or more treatment regimens based on the patient information and the guidelines. A physician or other healthcare provider can then evaluate the recommendations and select a treatment regimen for the patient. In a further embodiment, the Prescriptive Module 1207 selects the treatment regimen without selection advice from a health care provider. In an embodiment, the health care provider is able to review and modify the selection made by the Prescriptive Module. In another embodiment, the Prescriptive Module 1207 assists in evaluating one or more treatment regimens. For example, the Prescriptive Module 1207 can consider counter indications for a treatment regimen. In another embodiment, the Prescriptive Module 1207 assists a health care provider in titrating one or more medicaments included in a treatment regimen. In an embodiment, the Prescriptive Module 1207 recommends and/or selects a specific dosage of one or more medicaments to be given to the patient.

In an embodiment of the subject invention, the Dispensing Module 1209 evaluates a patient's compliance with a treatment regimen. In a further embodiment, the Dispensing Module 1209 transmits an indication of compliance or noncompliance with the treatment regimen to one or more persons involved in the care or treatment of the patient. As discussed above, persons involved in the care or treatment of the patient can include the patient, one or more health care providers treating the patient, and/or one or more payors financing the treatment of the patient, among other persons. The indication transmitted can serve various purposes and be received in various forms. For example, an alert message can be transmitted to the patient indicating that they forgot to take a medicament, or go to the gym, or take a test. In another embodiment, an alert message is sent to a health care provider indicating that the patient has repeatedly failed to take a medicament as directed. In another embodiment, an alert message is sent to a payor indicating that the patient has repeatedly failed to exercise as prescribed in the treatment regimen. In other embodiments, alert messages (or reminders) can be sent before a failure occurs, such as a reminder to take medication or go to the gym.

As discussed above, various communication technologies can be used with the subject invention to accomplish the transmission of such information. The subject invention is not limited to any particular communication technology. Communication can be over a network, such as the Internet, a LAN, WAN, VPN. Communication can occur via a mainframe or point-to-point connection. Various client devices can be used to send or receive such communication including, but not limited to, computers, PDAs, cellular phones, other client devices. Communication can also occur via various communication protocols known in the art. For example, various cellular communication technologies can be used. In an embodiment, Short Message Service (SMS) protocol is used. In another embodiment, email message protocols are used, such as Simple Mail Transfer Protocol (SMTP).

In an embodiment, the Dispensing Module 1209 utilizes a dispensing device, such as the Dispensing Device 1303 described below, to capture compliance information regarding a patient's taking of one or more medicaments. In an embodiment, the dispensing device includes sensors adapted to sense the presence, absence, or movement of one or more medicaments and/or their packaging so as to assess whether or not a patient has taken one or more medicaments. In a further embodiment, the dispensing device also includes a communication device adapted to transmit compliance information to one or more persons involved in the care or treatment of the patient. In a particular embodiment, the communication device transmits such compliance information to a server where it is processed and communicated on to one or more persons involved in the care or treatment of the patient.

In an embodiment, the Dispensing Module 1209 captures information regarding one or more tests, activities, or other treatments to be taken by the patient. For example, the Dispensing Module 1209 can receive blood glucose levels from a blood glucose meter, or the Dispensing Module 1209 can receive a message from a fitness center computer indicating that the patient entered or left an associated fitness center, or the Dispensing Module 1209 can receive a heart rate from a heart rate monitor worn by the patient or information from a pedometer worn or similar device worn by the patient. Other methods of monitoring patient compliance without action by the patient themselves can be used with the subject invention. In an alternative embodiment, the Dispensing Module 1209 receives compliance information from the patient themselves.

In an embodiment of the subject invention, the Feedback and Patient Management Module (“FPMM”) 1211 gathers compliance information and evaluates efficacy of a treatment regimen for a patient. In a further embodiment, the Feedback and Patient Management Module 1211 transmits information about the efficacy of the treatment regimen to one or more persons involved in the care or treatment of the patient. As discussed above, persons involved in the care or treatment of the patient can include the patient, one or more health care providers treating the patient, and/or one or more payors financing the treatment of the patient. In an embodiment, the Feedback and Patient Management Module 1211 tracks the patient's noncompliance, progress, and/or success with the treatment regimen. In an embodiment, the FPMM 1211 provides feedback to the patient regarding such noncompliance, progress, and/or success. In a further embodiment, the feedback provided to the patient is tailored for greater relevance to the patient's pattern of behavior, patient information, or other data. In an embodiment, the FPMM 1211 provides management advice to other persons involved in the patient's care or treatment regarding such noncompliance, progress, and/or success. In a further embodiment, the advice provided is tailored for greater relevance to the patient's pattern of behavior, patient information, or other data. In an embodiment, economic incentives or disincentives are offered to the patient based on such noncompliance, progress, and/or success. In another embodiment, the incentives or disincentives are mandatory. In an embodiment, reduced health premiums are offered for compliance with health diet or exercise activities required by the treatment regimen. In another embodiment, discounts on prescription drugs are offered for compliance with dosage requirements of the treatment regimen.

Various communications technologies can be used to deliver feedback and/or management advice to person in accordance with the subject invention. For example, such information can be delivered via mail, facsimile, email, automated telephone message, Internet Relay Chat, SMS message, website, newsgroup, or other post, among other possible communication methods. In an embodiment, persons select a preferred communication method from a set of available methods.

In an embodiment of the subject invention, information provided via one or more modules of the system 1201 causes an update to information in one or more other modules of the system thus creating a feedback loop. In another embodiment, such feedback occurs with human intervention. For example, a physician can receive management advice via the FPMM 1211 and decide to select a different treatment regimen via the Prescriptive Module 1207. In an embodiment, such feedback occurs electronically or independent of human intervention. For example, FPMM 1211 can automatically update patient information via the Diagnostic Module 1203 based on the patients progress. For example, the FPMM 1211 may update a patients weight received via the Dispensing Module 1209.

In an embodiment of the subject invention, the system 1201 includes a social networking component that allows persons involved in a patient's or patients' care or treatment to network. Various facilities can be used for such networking including, but not limited to, online communities, blogs, and other social media. In an embodiment, various networks are customized to the persons' particular needs. In an embodiment, such networks provide education, support, guidance, information, and/or motivation to the patient or other person involved in the patient's care. In a further embodiment, network members are suggested or selected by a FPMM, such as FPMM 1211. In an embodiment, various social networking actions and/or events can be prescribed as part of a treatment regimen.

In an embodiment of the subject invention, health clubs partner with person's involved in a patient's care or treatment. In an embodiment, such health clubs are provided financial or other incentives to partner. In a further embodiment, a portion of those incentives are passed on to the patient. In an embodiment, such health clubs are required to implement a reporting process by which patient compliance information is communicated to a FPMM, such as FPMM 1211. In a further embodiment, such patient compliance information is transmitted electronically via a communication device. Such compliance information can include, but is not limited to, attendance, progress, weight, bad-fat, and/or exercise activities of the patient, among other information.

In an embodiment of the subject invention, health food retailers and/or grocery stores partner with person's involved in a patient's care or treatment. In an embodiment, such stores are provided financial or other incentives to partner. In a further embodiment, a portion of those incentives are passed on to the patient. In an embodiment, such stores are required to implement a reporting process by which patient compliance information is communicated to a FPMM, such as FPMM 1211. In a further embodiment, such patient compliance information is transmitted electronically via a communication device. Such compliance information can include, but is not limited to, a record of purchase made by the patient at the store.

In an embodiment, a Disease Management System is provided comprised of five separate interrelated modules as shown in FIG. 12. In an embodiment, the Diagnostic Module exposes the patient and provides access to various disease management guidelines. The module can be accessed by a physician, care provider, or other user to obtain patient information or review guidelines for treatment. In an embodiment, patient information and/or guidelines are accessed via a database on a network. In an embodiment, the user—irrespective of his geographic location—has access to the patient's medical history in “real time,” and is augmented with scientific information from the National Institute of Health (NIH), the Center for Disease Prevention and Control (CDC), The American Heart, Lung and Blood Institute (AHLBI), The Food and Drug Administration (FDA), The American Institute of Radiologists, The American Diabetes Association (ADA), the World Health Organization (WHO), and/or other guidelines. In an embodiment, the database can be used by the physician, care provider, or other user for informed, educated, and experienced judgment about a patient's treatment.

In an embodiment, diagnostic evaluation is specifically directed at quantifying the probability of cardiovascular, cerebrovascular accidents, and the progression of insidious diabetic sequelae within a 10 year time frame, based on the NIH's Farmington Heart Study point evaluation methodology. In other embodiments, other timeframes or evaluative methodologies are used. In an embodiment, the quantification of diabetic sequelae contributing risk factors as a “Target Organ Disease” is incorporated within the module. In an embodiment, a patient's risk for cardiovascular and cerebrovascular accidents is accessed within the following categories: BELOW AVERAGE RISK, AVERAGE RISK, MODERATELY ABOVE AVERAGE RISK, HIGH RISK. The risk assessment methodologies and guidelines described herein are merely illustrative examples. Other systems for measurement for patient risk assessment and guidelines are known in the art and can be used with the subject invention.

### Example Scenario

The following is an example of how an embodiment of a Disease Management System can function. Other embodiments, may utilize more, fewer, or different components or functions. Also, the functions performed in this scenario are merely examples. Other components or functions are described below and can be used in a different order. For this scenario, assume the patient is a male, non-smoker who is 50 years of age with high overall cholesterol, moderate good cholesterol, and moderate to high blood pressure. In an embodiment, his coronary heart risk profile evaluation would score as follows:

TABLE 16

| Question or Condition | Answer | Score |
|---|---|---|
| Age | 50 | 3 |
| Total Cholesterol (240-279) | 255 | 2 |
| HDL Cholesterol (50-59) | 52 | 0 |
| Systolic Blood Pressure (140-159) | 145 | 2 |
| Diabetes | No | 0 |
| Smoker | No | 0 |

| | |
|---|---|
| Total Score | 7 |

A total score of “7” on the coronary disease risk profile from table 16 can then be plotted on the risk assessment table (Table 7 above) along the row indicated by the number 7, and intersecting with the column with an age heading of 50-54. Therefore, in this embodiment, this patient has a risk score of “2.6,” which indicates he has an “average risk” of CHD. The patient, with a risk score of 2.6, would then be prescribed a treatment regimen in accordance with the above matrix. In this scenario, the patient's quantified state of health data is advanced and correlated to a Prescriptive Module.

The Prescriptive Module, in this embodiment, includes guidelines from: the NIH, the CDC, the AHLBI, the FDA, the ADA, WHO, the Manufacturers Guidelines, and/or any other available guidelines. Using the Prescriptive Module, a customized prescription is manually and/or electronically computed for the patient from the patient information and guidelines, and/or further verified for potential adverse reactions against normal prescriptive guidelines, and/or electronically and/or manually transmitted to participants contributing to the patient's state of health (e.g., the patient's medical history file in the physician's database, the Pharmacy, the relevant Third-Party Payer (Insurance Company/Medicare/Medicaid), the patient's employer, the Agency maintaining the follow-up modality, and/or any other party). In an embodiment, the resulting prescription is delivered to the patient's home and/or any other location as further described below.

In an embodiment, a Dispensing Module is initiated with the receipt of a prescription from the patient's Physician, care provider, a Prescriptive Module, or other source. As further described below, the Dispensing Module can facilitate delivery of medicaments or other treatment components (e.g., motivational literature, educational literature, exercise equipment, scheduling or motivational messages, diet information, social networking contacts) to the patient. In a further embodiment, the Dispensing Module also receives compliance information back from the patient. Various compliance information can be collected with and/or without the assistance of the patient. In a further embodiment, the Dispensing Module transmits some or all of the compliance information to one or more persons involved in the patient's care or treatment. In a further embodiment, the Dispensing Module also evaluates the compliance information and can transmit a summary, highlights, or analysis of the compliance information to one or more persons involved in the patient's care or treatment.

In an embodiment, medicaments or other treatment components are delivered to the patient along with a dispensing apparatus. In an embodiment, the dispensing apparatus includes a dispensing device with sensors capable of sensing when the treatment components are moved, used, or not used for a selected length of time. Thus, compliance information can be derived from the dispensing apparatus. In an embodiment, such information is stored on the dispensing device or apparatus and later retrieved. In another embodiment, the dispensing apparatus comprises a communications device capable of sending such information to a computer on a network for further processing and/or transmission. The communication device can be incorporated into the body of the dispensing device or apparatus or as a separate component operably connected via a cable or other known means. In an embodiment, short-range wireless communications technologies, such as Bluetooth, can be used to connect the communication device to the dispensing device. As discussed above, various communication technologies can be used to accomplish transmission of information.

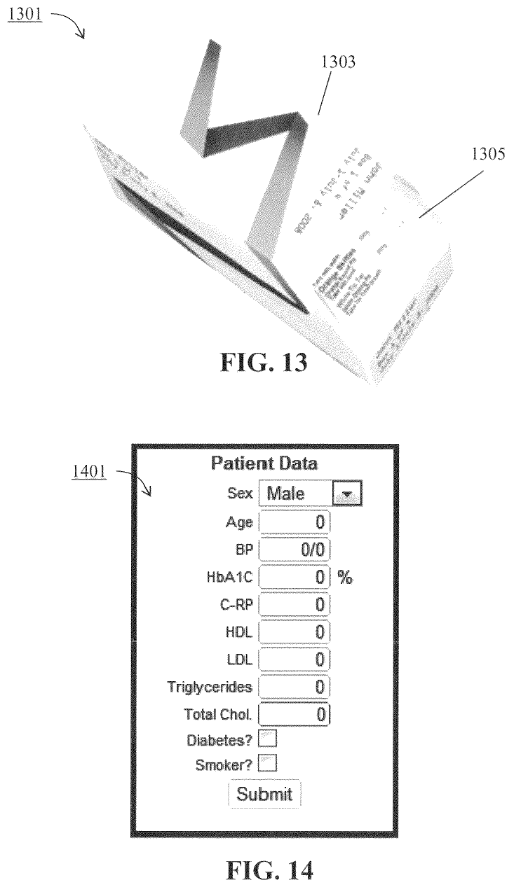

FIG. 13 illustrates an apparatus 1301 for dispensing medicaments in accordance with an embodiment of the subject invention. As shown, the apparatus includes a dispensing device 1303 and one or more packages 1305 for holding medicaments. In the embodiment shown, the dispensing device 1303 is shaped like a supply box. As discussed above, additional components can be incorporated into the body of such a supply box or connected to the supply box using various means known in the art. In an embodiment, the supply box has one or more ports or antennas on the back side of the device (not shown) for transmitting information. In an embodiment, the dispensing device 1303 includes one or more sensors capable of sensing movement or other information regarding the one or more packages 1305. For example, the movement of one or more packages 1305 can be determined by a change in weight. In an embodiment, the packages 1305 themselves comprise the sensors. In a further embodiment, the dispensing device 1303 includes a communication device for communicating information.

In an embodiment, the use or non-use of the dispensing device 1303 or the movement or non-movement of the one or more packages 1305 triggers the storage and/or communication of such an event or non-event (known as “compliance information”). In an embodiment, the trigger occurs when such an event or non-event happens within a specified timeframe. In another embodiment, the trigger occurs regardless of timeframe. As discussed above, the dispensing device 1303 can communicate compliance information using various communications technologies, including but not limited to: Cellular Phone Technology (Short Message Service—“SMS,” Global System for Mobile Communications—“GSM,” and/or any other technology utilizing the cellular communication platform), land based telephone or other hardwire linked (either connected or separate) to the communication device, and/or satellite communication technology.

In an embodiment, the dispensing device includes one or more indicators for presenting information to a user. The indicator can alert the user of various issues or conditions. For example, the indicator may indicate that it is time to take a prescribed action including, not limited to, taking a dose of medicament, exercising, taking a test, attending an appointment, eating a regular meal, among other prescribed actions. The indicator may also indicate failure to take such actions on time. In an embodiment, the indicator alerts a user to problem with the dispensing device including, but not limited to, that the device is low on batteries, that the device is low on medicaments, that the device is disconnected from a communications device, among other problems. In an embodiment, the indicator is displayed visibly, such as via an LED, textual display, or graphical display. In an embodiment, the indicator is presented audibly, such as via a speaker. In an embodiment, the indicator is connected to a communication device and is enabled to communicate such information to a computer on a network. Such a computer can thereafter notify one or more persons or entities involved in the care and treatment of the relevant patient. In an embodiment, the indicator can be reset, disabled, or “snoozed” via an input.

In an embodiment, a Patient Management Module, such as FPMM 1211, reviews data from the Patient Prescriptive Module, Dispensing Module, and/or any other source. In an embodiment, the Patient Management Module integrates electronic surveillance of the patient's prescription administration compliance with the use of the Internet, GSM technology, and human interaction with the patient. “Real Time” patient compliance/non-compliance data can be communicated to the physician, third party payers, the patient employer (if necessary), as well as the patient, and/or any other party. In an embodiment, the patient is reminded of missed doses. Health Club attendance, food purchases and consumption, and/or any other organization and/or activity that improves the patient's health (including but not limited to chronic disease[s]) and/or lowers the patient's health care costs can be electronically or by other means communicated to the Patient Management Module. In an embodiment, such information is further transmitted to relevant participants. In a further embodiment, the Patient Management Module evaluates and the degree of efficacy of the treatment regimen based on such information. In an embodiment, such measures and/or evaluations are communicated to relevant participants in real-time and/or any other time frame. In an embodiment, the progress and reduction of risk category is reinforced to the patient, third-party payers, employers, or any other relevant party that will advance the appropriate financial and/or other health awards and/or other motivation tools for the patient.

In an embodiment, a communication device known as a Patient Module Communication Device transmits the above described information to a recipient, a computer, a computer network or server, and/or non-networked servers, which may or may not utilize one or more intermediate communications and/or relaying technologies. Other communications means known in the art may be used with the subject invention.

### Patient Prescriptive and Monitoring Methodology

In an embodiment of the subject invention, persons involved in the patient's care or treatment have the option of using a manual and/or electronic data management system on a computer and/or non-computerized device that can be portable and/or non-portable called the Clinical Protocol Management Tool. This Clinical Protocol Management Tool utilizes and/or integrates information/data received from the Patient Module Communication Device specified above. This data management system may or may not provide data/information using charts, pictures, imaging, calendars, and/or any other method to communicate information that is inclusive but not limited to: patient disease management information (inclusive of but not limited to compliance) that includes, but not limited to, physician or other care giver's prescribed therapy (pharmaceuticals, medical imaging, consumables, exercise or other activity, or any other recommendation[s]).

In an embodiment, the Clinical Protocol Management Tool utilizes a scoring system and/or other systems of measurement for the patient's health risk factors. In an embodiment, the Clinical Protocol Management Tool provides a user of the Tool with a recommended treatment regime for a patient. In an embodiment, the treatment regime is given a specific name and/or symbol that may or may not be generated from the clinical protocol matrices for that combination and/or individual prescribed pharmaceuticals. In an embodiment, the user can use the Tool to change and/or customize and/or titrate dosages or other treatment regime components as indicated.

### Interfaces to the Disease Management System and Components

Next numerous example interfaces are described with reference to FIGS. 14-23C. These interfaces are merely examples. Embodiments of the subject invention can embody different interfaces comprising fewer, different, or additional components. Also the components depicted can be differently arranged and/or configured.

FIG. 14 illustrates an interface to a Diagnostic Module 1401 in accordance with an embodiment of the subject invention. In an embodiment of the subject invention, the interface 1401 can be used to input information about a patient.

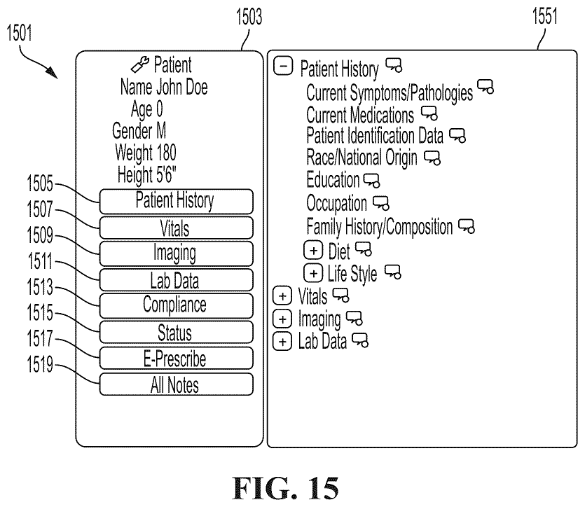

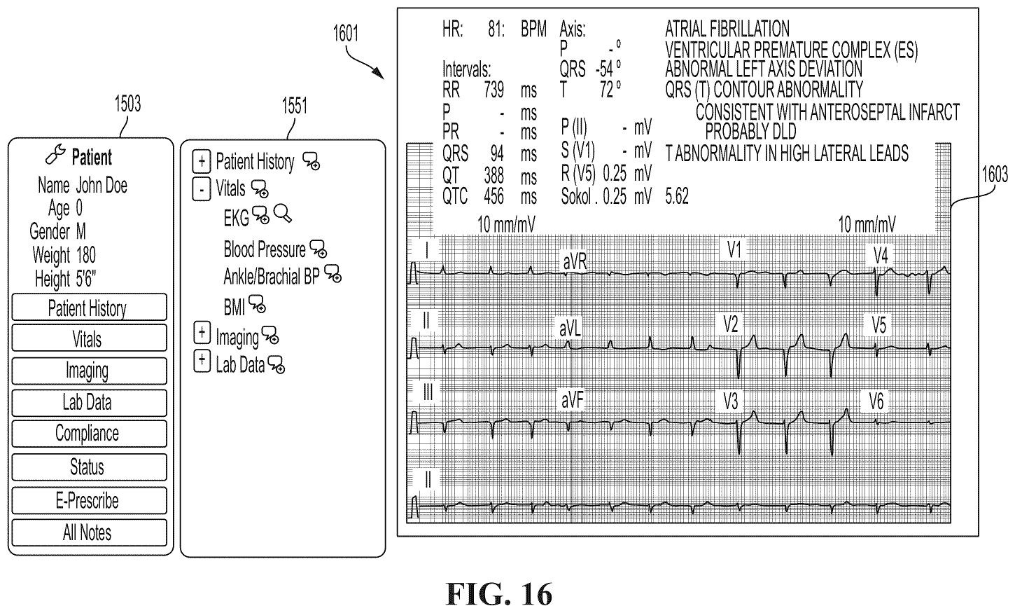

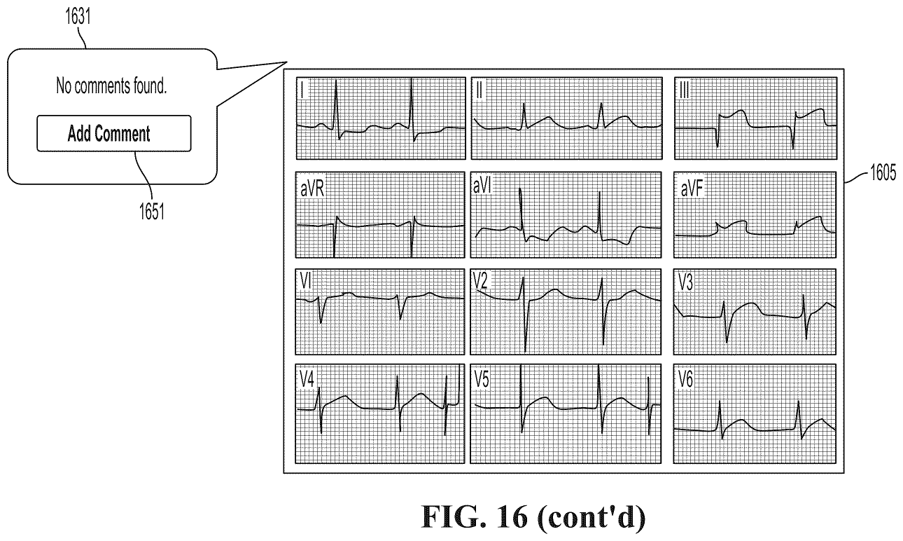

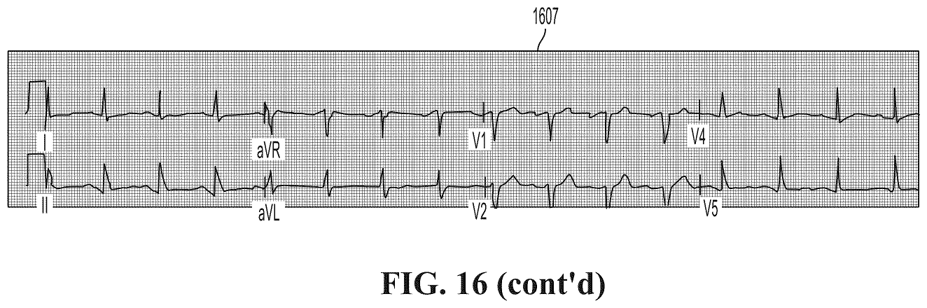

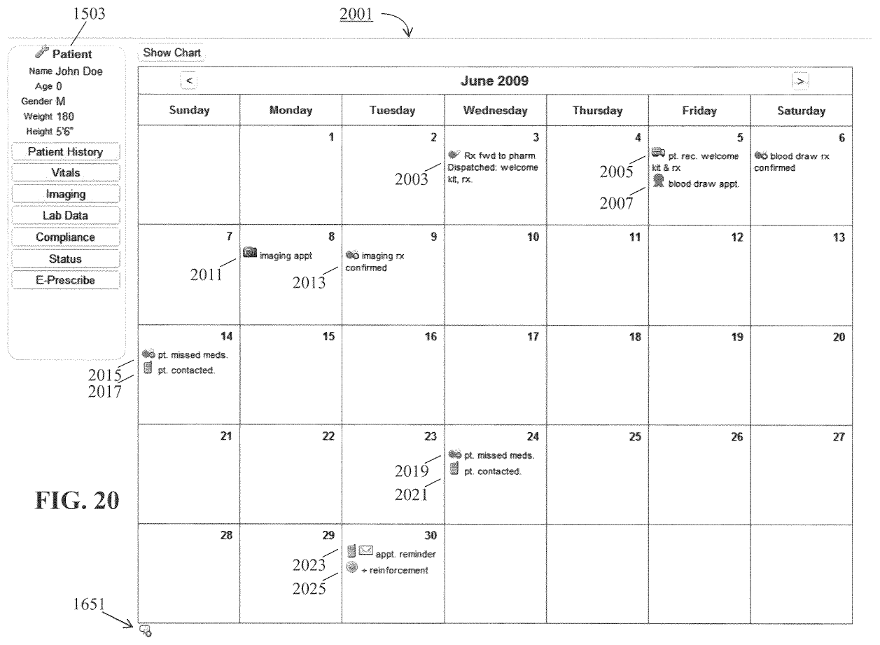

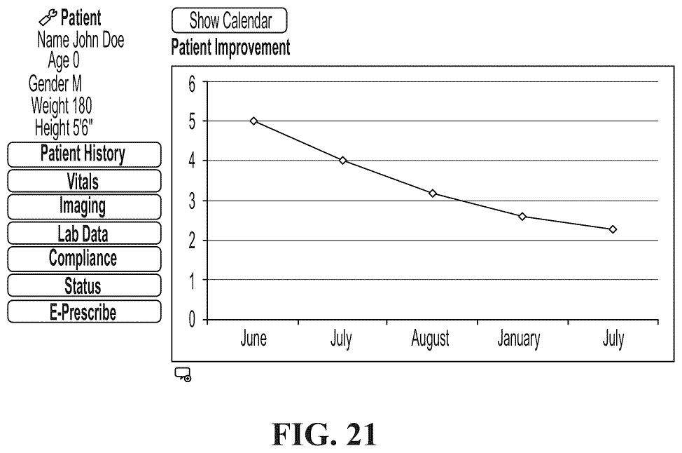

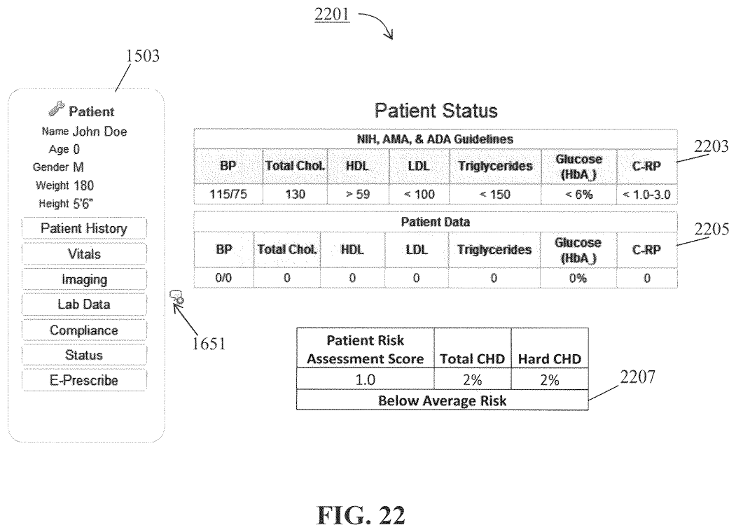

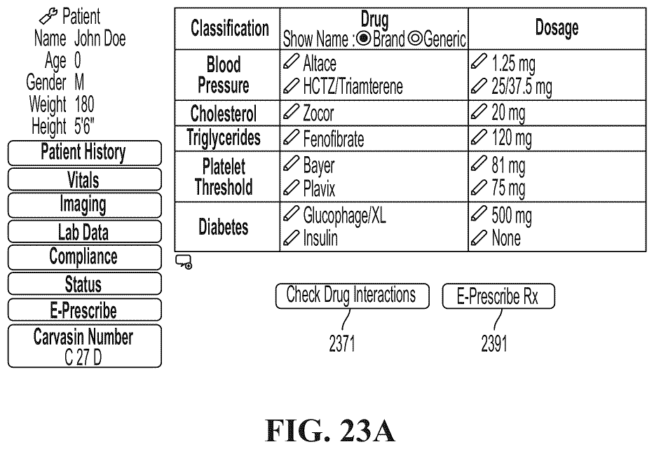

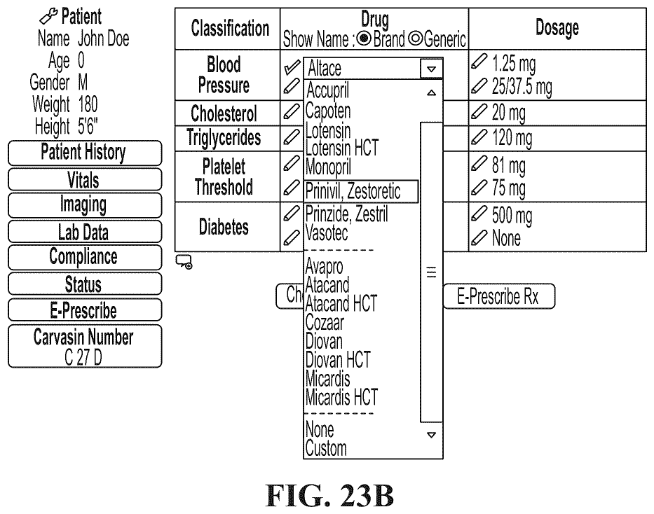

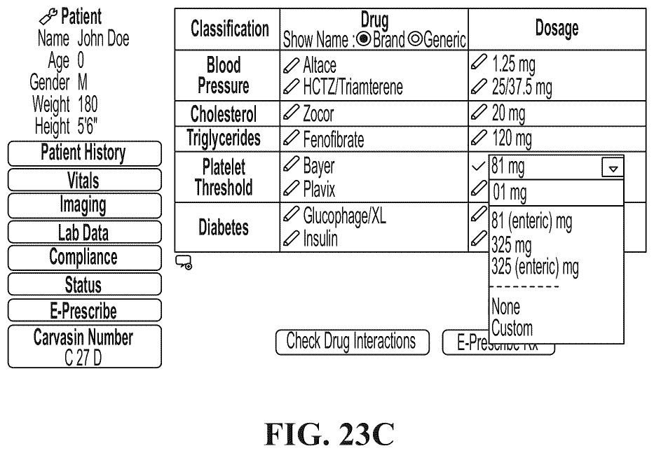

FIG. 15 illustrates an interface to a Disease Management System 1501 in accordance with an embodiment of the subject invention. In an embodiment of the subject invention, the interface 1501 can be used to access different components of a Disease Management System, such as the Disease Management System 1201. In the embodiment shown, a Disease navigation pane 1503 is provided. A user of the Disease Management System can use the navigation pane 1503 to access different components of the Disease Management System. For example, buttons 1505-1511 can provide access to different interfaces to a Diagnostic Module, such as the Diagnostic Module 1203. The embodiment shown also includes a patient chart interface 1551. As further illustrated in FIGS. 16-19, the patient chart interface 1551 can be used to access different interfaces to patient information within the Diagnostic Module. In FIG. 15, the “Patient History” portion of the patient chart interface 1551 is expanded to allow access to various patient history information. In an embodiment, button 1513 provides access to an interface to a Feedback and Patient Management Module, such as FPMM 1211. Example embodiments of such an interface are described below with reference to FIGS. 20-21. In an embodiment, button 1515 provides access to an interface to a Diagnostic Interpretive Module, such as Diagnostic Interpretive Module 1205. An example embodiment of such an interface is described below with reference to FIG. 22. In an embodiment, button 1517 provides access to an interface to a Prescriptive Module, such as Prescriptive Module 1207. An example embodiment of such an interface is described below with reference to FIGS. 23A-C.

In an embodiment of the subject invention, notes or comments can be added or viewed by various users of a Disease Management System. These notes are appropriate for commenting on various data or results presented by such a system. In an embodiment, notes can be attached to the different components of the Disease Management System. In an embodiment, “global” notes can also be associated with the system as a whole. In the embodiment shown in FIG. 15 button 1519 provides access to all such notes regardless of the portion of the system they are associated with.

FIG. 16 illustrates an interface to a Diagnostic Module 1601 in accordance with an embodiment of the subject invention. In the embodiment shown, interface 1601 presents various patient information related to a patient's clinical status (e.g., 1603, 1605, 1607). In the embodiment shown, the patient chart interface 1551 can be used to gain access to EKG, Blood Pressure, Ankle/Brachial BP, and BMI. Window 1631 illustrates an example of a note field that can be used to comment on the information presented. In this example, no notes are available. In an embodiment, button 1651 can be used to add a note.

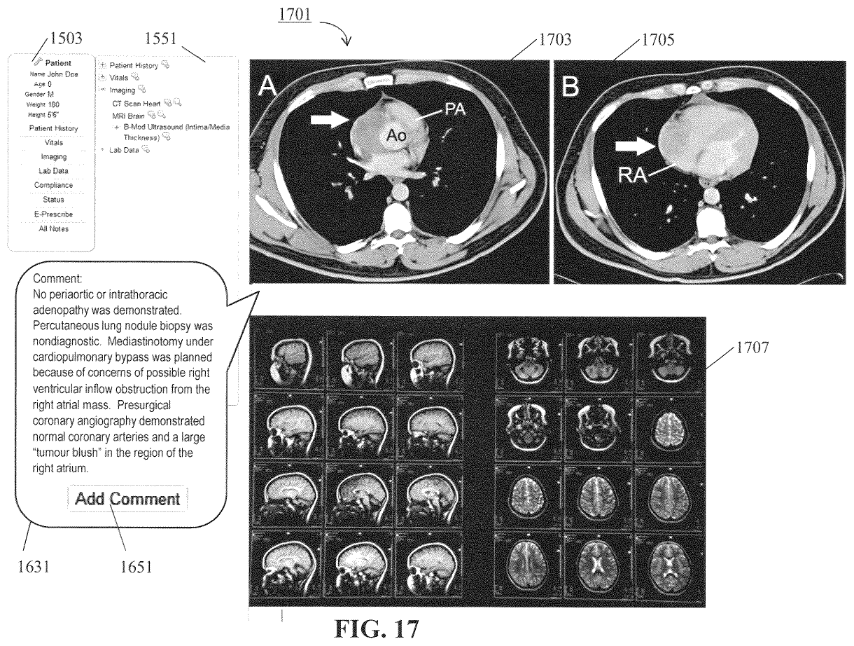

FIG. 17 illustrates an interface to a Diagnostic Module 1701 in accordance with an embodiment of the subject invention. In the embodiment shown, interface 1701 displays various imaging of a patient (e.g., 1703-1707). In the embodiment shown, the patient chart interface 1551 can be used to gain access to additional imaging. Window 1631 illustrates an example of a note field with a comment related to image 1703. In this example, button 1651 can be used to add additional notes.

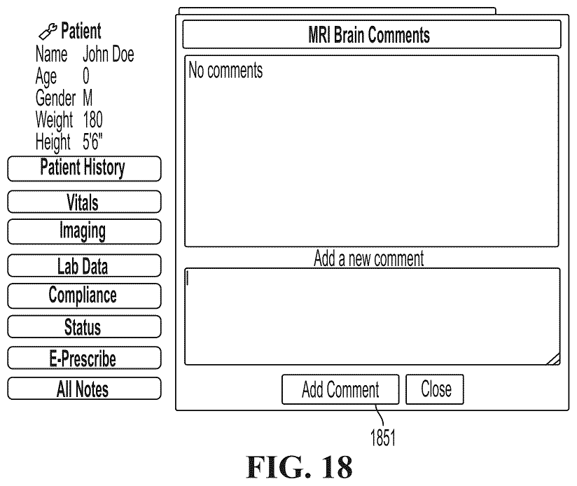

FIG. 18 illustrates an interface to a Diagnostic Module 1801 in accordance with an embodiment of the subject invention. In the embodiment shown, interface 1801 presents various information related to a Brain MRI of a patient. Window 1831 can be used to comment on the Brain MRI. In an embodiment, button 1851 can be used to add such a comment.

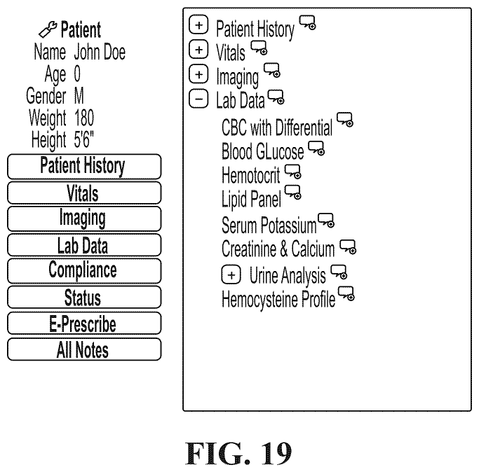

FIG. 19 illustrates an interface to a Diagnostic Module 1901 in accordance with an embodiment of the subject invention. In the embodiment shown, interface 1901 allows access to various patient information related to a patient's lab data. In the embodiment shown, the patient chart interface 1551 can be used to gain access to various lab data.

FIG. 20 illustrates an interface to a Feedback and Patient Management Module 2001 in accordance with an embodiment of the subject invention. In the embodiment shown, interface 2001 displays information regarding a treatment regimen prescribed for a patient. In the embodiment shown, this information is displayed on a calendar interface; however, various other interfaces can be used to display such information including a task or other list, a timeline, among other known interfaces. In the embodiment shown, interface 2001 displays both prescriptive and descriptive information about the treatment regimen. For example, interface 2001 includes prescriptive information about tests or other events to be performed by the patient or others as part of the treatment regimen. In example displayed, the patient is to have a blood draw appointment on Saturday, Jun. 6, 2009 (2007) and an imaging appointment on Monday, Jun. 8, 2009 (2011). These are merely examples other types of tests or other events can be prescribed as part of a treatment regime and displayed on the interface 2001. In an embodiment, the interface 2001 can also show actions to be taken by other persons or entities involved in the patient's care. For example, on Tuesday, Jun. 30, 2009, the patient is to be reminded of an appointment (2023). This reminder can be made by a healthcare provider or other person. In another embodiment, the reminder is electronically performed via a FPMM, such as FPMM 1211, without human involvement. Interface 2001 also presents descriptive information, which describes events related to the patient's treatment that occurred or failed to occur as prescribed. As shown, some of the events can describe actions or omissions of the patient; others can describe actions or omissions of others. In the example displayed, a prescription was sent to a pharmacy and welcome kit was dispatched on June 3^{rd} (2003). As further discussed above, the treatment regimen can include various motivational, educational, or other literature provided to the patient. A welcome kit is an example of such literature. In an embodiment, the literature is customized for the patient by a FPMM, such as FPMM 1211. In a further embodiment, the welcome kit introduces the patient to a social networking component of the treatment regimen as further described above. Also in the displayed example, a note 2005 indicates that the patient received the prescribed medicaments and the welcome kit on June 5^{th} and a note 2013 indicates that the imaging appointment on June 8^{th} was confirmed. As shown, the interface 2001 can also indicate omitted actions. For example, the interface 2001 indicates the patient missed taking one or more medicaments on June 14^{th} and June 24^{th} (2015, 2019). As discussed above, various alert messages can be sent to the patient to remind the patient to perform various actions prescribed by the treatment regimen. The interface 2001 shows that the patient was alerted to the missed doses on the same day (2017, 2021). In the displayed example, note 2025 indicates that positive reinforcement information was sent to the patient on June 30^{th}. Positive reinforcement information is an example of customized literature provided to the patient by a FPMM or other entity. In the embodiment shown, button 1651, described above, has a different appearance but still performs the same functions.

FIG. 21 illustrates an interface to a Feedback and Patient Management Module 2101 in accordance with an embodiment of the subject invention. In the embodiment shown, interface 2101 displays information regarding a patient's results related to a treatment regimen. Results can be displayed since initiation of the treatment regimen, since before initiation, for a subset date range, or other time period. In the embodiment shown, button 2103 provides access to a calendar interface, such as calendar interface 2001. Also in the embodiment show, an improvement chart 2105 is displayed. Various charts can be used presenting various results data for the patient. In the example shown, the patient's overall risk score is plotted over time. This type of information can motivate the patient to continue to improve. In an embodiment, this type of information is included in motivational literature as described above.

FIG. 22 illustrates an interface to a Diagnostic Interpretive Module 2201 in accordance with an embodiment of the subject invention. In the embodiment shown, interface 2201 displays guidelines for various risk factors (2203) and patient information corresponding to the risk factors displayed (2205). In the embodiment shown, interface 2201 also displays a risk score (2207) calculated from the risk factors. As discussed above, various guidelines, risk factors, and risk assessment methodologies can be used with the subject invention. In the embodiment shown, the risk score is further categorized into a risk category (2207). Estimates of absolute risk of CHD are also displayed (2207). Absolute risk is expressed as a percentage likelihood of developing CHD per decade. Total CHD risk equates to all forms of clinical CHD, whereas hard CHD includes clinical evidence of myocardial infarction and coronary death.

FIGS. 23A-C illustrate an interface to a Prescriptive Module 2301 in accordance with an embodiment of the subject invention. In the embodiment shown, interface 2301 displays various drug classifications (2303), drugs within those classifications (2305), and available dosages of the drugs (2321). As shown in FIGS. 23B and 23C respectively, drop down menus are provided to allow a user to select a particular drug (2309) and a corresponding dosage (2323). Also in the embodiment shown, a radio-button 2307 can be used to change the interface to display generic names for the various drugs. Once a drug and dosage has been chosen, an indicia 2351 can be displayed corresponding to the selected treatment. As described above, various indicia can be used with the subject invention to indicate various medicaments and/or treatment regimens. In a further embodiment, other treatment components (e.g., exercise, diets, testing) can be selected via an expanded interface (not shown). In the embodiment shown, a check drug interactions button 2371 is provided. In an embodiment, button 2371 access a function which checks for counter-indications of the selected treatment regimen as described above. If counter-indications exist, they can be presented via the interface 2301 (not shown). In the embodiment shown, a an E-Prescribe Drug button 2391 is also provided. In an embodiment, button 2391 initiates delivery of components of the selected treatment regimen to the patient. In a further embodiment, such delivery is initiated electronically without additional human intervention. In an embodiment, such delivery is accomplished via the method 1001. The above-described methods, systems, interfaces, and data structures can be implemented as computer-readable code in one or more computer-readable media. As is known in the art, data and instructions can be stored in a single computer-readable medium or distributed amongst multiple computer-readable media.

The claims appended hereto are meant to cover modifications and changes within the scope and spirit of the present invention.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

It should be understood that any reference in this specification to “one embodiment,” “an embodiment,” “example embodiment,” “further embodiment,” “alternative embodiment,” etc., is for literary convenience. The implication is that any particular feature, structure, or characteristic described in connection with such an embodiment is included in at least one embodiment of the invention. The appearance of such phrases in various places in the specification does not necessarily refer to the same embodiment. Further, when a particular feature, structure, or characteristic is described in connection with any embodiment, it is submitted that it is within the purview of one skilled in the art to affect such feature, structure, or characteristic in connection with other ones of the embodiments.

The invention has been described herein in considerable detail, in order to comply with the Patent Statutes and to provide those skilled in the art with information needed to apply the novel principles, and to construct and use such specialized components as are required. However, it is to be understood that the invention can be carried out by specifically different equipment and devices, and that various modifications, both as to equipment details and operating procedures can be effected without departing from the scope of the invention itself. All or some of the embodiments can be selectively combined to yield variants. Many different arrangements of the various components depicted, as well as components not shown, are possible without departing from the spirit and scope of the present invention. A skilled artisan can develop alternative means of implementing the aforementioned improvements without departing from the scope of the present invention. It will be understood that certain features and subcombinations are of utility and can be employed without reference to other features and subcombinations and are contemplated within the scope of the claims. Not all steps listed in the various figures need be carried out in the specific order described. Further, it should be understood that, although the present invention has been described with reference to specific details of certain embodiments thereof, it is not intended that such details should be regarded as limitations upon the scope of the invention except as and to the extent that they are included in the accompanying claims.

We claim:

1. A multi-unit dose package (MUDP) for containing a plurality of different pill medications, the package comprising:
a container zone with at least two distinct compartments;
each of said compartments separated from another by a thin partition; and
a plurality of pills, each pill a different medicament and each of said compartments containing at least one pill, said plurality of pills constituting a multi-unit dose,
wherein each of said compartments of the container zone is pie shaped such that, when viewed from above, said container zone appears to have a circular circumference, wherein the thin partition is disposed at segment boundaries between each adjacent pie shaped compartment of the container zone.

2. The MUDP of claim 1, wherein the plurality of pills comprises an aspirin or a PLA VIX, and at least one pill-form medicament selected from the group consisting of a diuretic, an ACE inhibitor, a statin, a triglyceride inhibitor, and a metformin.

3. The MUDP of claim 2, wherein the MUDP is one of a plurality of MUDPs packaged into a multi-day supply container.

4. A disease management system, comprising:
a selection system for selecting a treatment regimen based on an evaluation of a patient, wherein the selection system comprises:
an initial diagnostic module interface having data input fields for inputting information about a patient, the data input fields including fields for information including sex, age, and profile or test results,
a disease management system interface displaying on a screen after information is submitted via the initial diagnostic module interface, wherein the disease management system interface comprises a navigation pane, the navigation pane providing access to disease management system components including patient history information, compliance information, and status information,
a diagnostic interpretive module receiving the information input to the data input fields of the initial diagnostic module interface and using the information to provide and display a patient risk assessment score according to a categorized level of risk for particular diseases via a diagnostic interpretive module interface, the diagnostic interpretive module also displaying risk factor guidelines, and
a prescriptive module selecting the treatment regimen using the patient risk assessment score from the diagnostic interpretive module.

5. The disease management system according to claim 4, wherein selection of the patient history information in the navigation pane opens a patient chart interface for accessing patient information, the patient chart interface being presented on a same screen as the navigation pane, wherein the patient chart interface for accessing patient information comprises at least one field note data entry selection, wherein the field note data entry selection opens a note field in a window overlying a portion of the screen displaying the patient chart interface and the navigation pane.

6. The disease management system according to claim 5, wherein the patient chart interface comprises a selection for vitals, wherein the selection for vitals opens, in a same window as the patient chart interface and the navigation pane, various patient information related to a patient's clinical status including an EKG plot.

7. The disease management system according to claim 5, wherein the patient chart interface comprises a selection for imaging, wherein the selection for imaging opens, in a same window as the patient chart interface and the navigation pane, various imaging of the patient.

8. The disease management system according to claim 5, wherein the patient chart interface comprises a selection for lab data.

9. The disease management system according to claim 4, wherein selection of the compliance information in the navigation pane opens a feedback and patient management module interface, the feedback and patient management module interface displaying information regarding a treatment regimen prescribed for a patient.

10. The disease management system according to claim 9, wherein the feedback and patient management module interface comprises a calendar or timeline interface.

11. The disease management system according to claim 9, wherein the feedback and patient management module interface comprises a screen displaying information regarding a patient's results related to a treatment regimen.

12. The disease management system according to claim 9, further comprising:
a dispensing module for dispensing the selected treatment regimen to the patient and communicating compliance information to the selection system.

13. The disease management system according to claim 12, wherein the selection system further comprises a feedback and patient management module that receives the compliance information from the dispensing module.

14. The disease management system according to claim 13, wherein the feedback and patient management module communicates information regarding a patient's compliance with the selected treatment regimen to a person involved in the care or treatment of the patient.

15. The disease management system according to claim 14, wherein the feedback and patient management module also communicates information regarding a patient's results with the selected treatment regimen to the person involved in the care or treatment of the patient.

16. The disease management system according to claim 12, wherein the diagnostic interpretive module further receives information from the feedback and patient management module in order to reevaluate and provide an updated assessment score.

17. The disease management system according to claim 16, wherein the prescriptive module selects the treatment regimen using the updated assessment score from the diagnostic interpretive module.

18. The disease management system according to claim 13, wherein the prescriptive module further receives information from the feedback and patient management module and selects a new treatment regimen according to the received information.

19. The disease management system according to claim 4, wherein selection of the status information in the navigation pane opens the diagnostic interpretive module interface, the diagnostic interpretive module interface being presented on a same screen as the navigation pane.

20. The disease management system according to claim 4, wherein the navigation pane further provides access to prescription information, wherein selection of the prescription information in the navigation pane opens a prescriptive module interface, the prescriptive module interface being presented on a same screen as the navigation pane.

21. The disease management system according to claim 20, wherein the prescriptive module interface displays drug classifications, corresponding drugs, and available dosages.
